(19) United States
(12) Patent Application Publication
(10) Pub. No.: US 2002/0098246 A1
(43) Pub. Date: Jul. 25, 2002

(54) Compositions, methods, apparatuses, and systems for singlet oxygen delivery

(76) Inventor: Randolph M. Howes, Kentwood, LA (US)

Correspondence Address: FINNEGAN, HENDERSON, FARABOW, GARRETT &, DUNNER LLP, 1300 I STREET, NW, WASHINGTON, DC, 20005, US

(21) Appl. No.: 10/050,121

(22) Filed: Jan. 18, 2002

Related U.S. Application Data
(63) Continuation-in-part of application No. 10/023,754, filed on Dec. 21, 2001.
(60) Provisional application No. 60/262,635, filed on Jan. 22, 2001.

(51) Int. Cl. A61K 33/40

(52) U.S. Cl. 424/613

(57) ABSTRACT

Methods of treating tumors, lesions, and cancers comprising delivering to the affected site a combination of peroxide and hypochlorite anion. Hydrogen peroxide and sodium hypochlorite are possible sources of peroxide and hypochlorite anion, respectively. The reactants may be injected simultaneously or sequentially, and combine at the site to produce singlet oxygen. Singlet oxygen may be delivered to the treatment site or generated at the treatment site.

### RELATED APPLICATIONS

This application is a continuation-in-part of a U.S. Application entitled COMPOSITIONS, METHODS, APPARATUSES, AND SYSTEMS FOR SINGLET OXYGEN DELIVERY, filed Dec. 21, 2001 in the name of Randolph M. Howes, and further claims priority under 35 U.S.C. §119(e) to U.S. Provisional Application No. 60/262,635, filed Jan. 22, 2001, the entire disclosure of each of which is incorporated herein by reference.

### FIELD OF THE INVENTION

The present invention relates to methods, apparatuses, and systems for singlet oxygen delivery. In particular, the present invention relates to methods of providing singlet oxygen delivery comprising administering a source of peroxide and a source of hypochlorite, as well as systems and apparatuses for use in the method. The source of peroxide may be hydrogen peroxide, and the source of hypochlorite may be sodium hypochlorite.

### BACKGROUND

Control and destruction of unwanted living organisms is a critical part of healthcare throughout the world. Pathogens, such as bacteria, viruses, fungi, single and multicellular organisms, which normally live outside a person, can become destructive or life-threatening if allowed to take hold and reproduce in or on a person. Enormous resources, in the United States and abroad, are allocated to the control and destruction of such pathogens.

Sterilants and disinfectants may be considered a first line of defense, killing pathogens in an environment outside a living body. These products are intended to kill pathogens before they ever have an opportunity to contact a person and generate an infection. Household and industrial cleaning products are well known examples that frequently include an antimicrobial agent to reduce the population of pathogens. Traditionally, such products have been important for use in areas of food preparation or consumption, such as kitchens or restaurants, and in areas where pathogens are more likely to be found, such as bathrooms or locker rooms. Sterilants and disinfectants have been especially important in areas in which control of pathogens is critical, such as in medical treatment facilities, including veterinary and human facilities, hospitals, and in particular, operating rooms in such facilities. More recently, and in particular following the recent events in the United States, sterilants and disinfectants have been used to decontaminate areas that have been exposed to biological weapons such as anthrax.

Generally, sterilants and disinfectants are toxic or corrosive and thus can only be applied to inert surfaces, not directly to people or animals. That is, their toxicity generally precludes their application directly onto people or animals, where the toxicity would be too great. However, other compositions that may be applied directly to people and animals do exist and are commonly used. These compositions are often referred to as antiseptics.

Antiseptic agents are generally used in controlling or reducing the population of pathogens that have already contacted a living being, or in areas where prophylaxis is important. For example, topical antiseptics are applied to skin abrasions and wounds to prevent infection. Antiseptics are also formulated in washes, such as in shampoos, soaps, or detergents, which may be used to topically reduce and control pathogen population. Antiseptic formulations, however, are generally too toxic to be taken internally by humans or animals.

Antibiotic compositions may be administered to humans and animals. Such compositions generally exhibit a high degree of pathogen toxicity, yet are formulated to minimize human and animal toxicity. These compositions can be used when pathogens breach a body's protective defenses. Diseases produced by pathogens are well known, as are the antibiotics often used in their treatment. Antibiotics, such as dactinomycin, daunorubicin, doxorubicin, and the bleomycins, have also been used in treating diseases such as cancer by targeting abnormally proliferating cells.

Cancer remains one of the leading causes of death in the United States and the world. Treatment of cancer focuses on killing cancerous cells, yet avoiding the significant side effect of death to surrounding healthy cells. While improvements have been made in the area of cancer treatment, surgery, radiotherapy, and chemotherapy, each is still associated with significant side effects and limitations. And the side effects, such as toxicity and immunosupression, often further contribute to patient illness and hamper the patient's ability to recover. Thus, efforts at developing new treatments aim to maximize effectiveness while minimizing side effects and reduce the overall worldwide cancer death rate.

A newer method that has had some success in maximizing effectiveness and minimizing side effects is photodynamic therapy. This treatment generally involves infusing a photoactive compound into a patient and allowing the compound to collect in a tumor that is to be targeted. The photoactive compound in the tumor is irradiated with light energy (photons), thereby generating the killing compound, which is a short-lived oxygen specie called electronically excited singlet oxygen. The singlet oxygen is believed to produce toxic effects on the cells of the tumor through oxidation and/or free radical reactions. Photodynamic therapy has been effective in treating multiple types of cancer, including cancers of different tissues and organs, including benign and malignant tumors.

A similar technique has been used in the treatment of atherosclerosis, which is a type of arteriosclerosis. The word “atherosclerosis” comes from the Greek words athero, meaning gruel or paste, and sclerosis, meaning hardness. The disease results from deposits of fatty substances, including cholesterol and cholesterol esters, as well as cellular waste products, calcium, and other substances on the inner lining of an artery. This build-up is called a plaque, and such plaques may grow large enough to significantly reduce blood flow through an artery and produce major ischemic problems, including stroke and/or death. The plaques can also become fragile and weaken vascular walls or produce microemboli.

Past attempts to prevent or treat damage caused by atherosclerosis included, for example, coronary artery bypass surgery, mechanical or laser plaque removal, balloon angioplasty, and placement of scaffolding stents. More recently, photodynamic therapy has been suggested as an alternative therapy. (See, for example, the news release dated Sep. 4, 2001, by Pharmacyclics, Inc. reported to the 23^{rd} Congress of the European Society of Cardiology, noting that Phase I clinical trials of photoangioplasty with Antrin (motexafin lutetium) was feasible and well tolerated.)

Photoactive compounds are useful because of their ability to produce singlet oxygen by absorbing light energy and becoming unstable. In their unstable form, photoactive compounds interact with oxygen to excite it from its stable triplet electron state to its excited singlet state, i.e., to singlet oxygen (¹O₂*). The singlet oxygen then produces the desired effect on the target area, be it cancer cells or atherosclerotic plaque tissue.

The efficient production of singlet oxygen using photodynamic therapy, thus, requires the presence of molecular oxygen at the target site. While this is not problematic at the beginning of the photodynamic reaction, it becomes problematic as the reaction progresses and oxygen is consumed and blood vessels to the area thrombose. As the reaction depletes oxygen in the target area, the reaction rate is reduced. And as the reaction entirely depletes oxygen from the target tissue, the reaction entirely ceases to produce the desired end product, singlet oxygen. Once in this anoxic state, the tissue is not further affected by the photodynamic therapy, other than by the undesirable side effects of residual photosensitizer compounds.

Attempts to overcome this limitation have included cycling the irradiation with light, i.e., periods of light exposure followed by periods of dark, thereby allowing ground state molecular oxygen to diffuse into the target tissue following a reaction period and allowing the reaction to reoccur. Oxygen loading, another technique, attempts to increase oxygen concentration in the patient's blood through use of hyperbaric conditions. Thus, oxygen is enriched at the tumor site, and the photodynamic effect is initially enhanced. However, as both of these methods merely provide temporary solutions, neither truly solves the drawbacks of photodynamic therapy.

Another difficulty in photodynamic therapy arises from the fact that a photoactive agent is injected into the body and then left to circulate. While it is desirable that the compound collect in the tumor tissue, this effect varies between individual patients. It is thus difficult to determine the appropriate light energy to be applied when the amount of photoactive agent varies between patients, tissues, and/or cell types. Also, because the agent is left to circulate in the body, significant photosensitivity occurs. Thus, untargeted portions of the body are unintentionally treated upon exposure to sunlight.

Photodynamic therapy is also limited by the need for a highly focused, light-generating system, which is usually provided by a laser. Laser penetration of tissue is limited to approximately 3 centimeters, making large or deep tissue tumors more difficult to treat with photodynamic therapy. Also, although laser use has become very common in medical applications, some technical expertise in laser operation is still necessary. Moreover, medical-quality lasers, even small ones, can be expensive. There is, therefore, a need in the art for a method of delivering singlet oxygen to a tumor target without the drawbacks associated with photodynamic therapy.

As a molecular specie, the existence of singlet oxygen has been recognized for years. In 1939, Kautsky (Trans. Faraday Soc. 35:216) proposed that an excited form of oxygen might be responsible for photooxidation reactions. Chemical studies that supported Kautsky's hypothesis were performed in the 1960s. Using a peroxide-hypochlorite anion system, Foote and Wexler (J. Amer. Chem. Soc. 86:3879 (1964)) demonstrated that products generated, including singlet oxygen, were identical to those obtained through dye-sensitized photooxidation. This reaction of hydrogen peroxide and hypochlorite to produce singlet oxygen was important in many of the early studies of singlet oxygen.

The reaction causes the decomposition of one molecule of hydrogen peroxide into one molecule of singlet oxygen and water. The reaction is shown below:

H₂O₂+ClO⁻→¹O₂*+Cl⁻+H₂O

Singlet oxygen is not foreign to the human body. Early work in the field by Howes and Steele (Res. Commun. Chem. Pathol. Pharmacol. 2:619-626 (1971); Res. Commun. Chem. Pathol. Pharmacol. 3:349-357 (1972)) suggested a possible involvement of singlet oxygen in liver microsomal hydroxylation reactions. Today, singlet oxygen is recognized as the principle bacterial oxidizing agent employed by the human neutrophil (macrophage) and monocyte phagosome. Although not entirely understood, it is believed that myeloperoxidase, hydrogen peroxide, and chloride combine to produce powerful oxidizing compounds, including singlet oxygen, in the phagosome. It has been proposed that the myeloperoxidase reacts with the hydrogen peroxide to form the singlet oxygen and hypochlorous acid.

The present invention takes advantage of the reaction between peroxide and hypochlorite to produce singlet oxygen. The present invention solves the aforementioned problems in photodynamic therapy, and also finds use in treating, for example, tumors and atherosclerotic plaques. And because its components are naturally occurring and safe, yet capable of a potent oxidizing potential, the present invention also finds use as a sterilant, disinfectant, antiseptic, and antibiotic.

### SUMMARY OF THE INVENTION

### Features and Advantages of the Invention

This invention is advantageous in providing compositions, methods, apparatuses, and systems, for producing singlet oxygen.

It is advantageous that the singlet oxygen may be produced using chemical entities that are physiologically produced and physiologically present, without the need to resort to complex synthetic compounds, many of which have toxic or harmful side effects. The invention is advantageous in that the compositions are easily metabolized by body's natural metabolic mechanisms.

This invention may be used as a disinfectant, decontaminating agent, containment agent, sterilant, antiseptic, and antibiotic, and may be used on inert surfaces, as well as topically or internally for living animals, including humans.

The invention may be used in decontaminating areas exposed to chemical or biological agents.

When used inside a living animal, this invention may be used to target the therapy at the desired site, without exposing the entire patient or the surrounding tissue to collateral damage, and the reactants and products decompose into well tolerated physiological compounds.

This invention is also advantageous in providing singlet oxygen therapy to a site in need of therapy, using only simple surgical techniques, and without the need for expensive electronic equipment.

Additionally, the chemical constituents can be accurately regulated by concentration, rate of infusion, or infiltration and by precise depth of penetration.

It is also advantageous in that it does not have a limited depth of penetration and can be accurately administered at any desirable depth.

It is also advantageous that this invention may be repeatedly administered without undue effects.

### SUMMARY OF THE INVENTION

The present invention is directed to methods of treating a target site in or on a mammal, comprising administering a source of singlet oxygen, which may comprise administering at least one source of peroxide and at least one source of hypochlorite anion to the target site to be treated and allowing the peroxide and hypochlorite to react to produce singlet oxygen. In some embodiments, the source of peroxide comprises at least one of hydrogen peroxide, alkyl hydroperoxides, or metal peroxides.

In this invention, the source of hypochlorite anion may comprise at least one of metal hypochlorites or hypochlorous acid. Metal hypochlorites may be chosen from calcium hypochlorite, sodium hypochlorite, lithium hypochlorite, and potassium hypochlorite. The hypochlorite anion source may comprise chlorine dioxide.

In the present methods, the source of peroxide and source of hypochlorite anion may be administered sequentially. The source of peroxide and source of hypochlorite anion may be administered through at least one conventional syringe and needle. In the present invention, the source of peroxide and source of hypochlorite anion may also be administered simultaneously. In some embodiments, the source of peroxide and source of hypochlorite may be delivered through at least one dual lumen catheter.

The methods of the invention may be used where the target site is a tumor or an atherosclerotic plaque. The administration may be performed such that the source of peroxide and/or the source of hypochlorite anion is delivered upstream of blood flow to the target site and the blood flow carries at least one of the source of peroxide and the source of hypochlorite anion to the target site.

The invention is also directed to singlet oxygen produced by processes comprising a) introducing into a mammal at least one composition comprising at least one source of peroxide; and b) introducing into a mammal at least one composition comprising at least one source of hypochlorite anion.

This invention is also directed to systems for treating a target site in a mammal, comprising a) at least one source of peroxide; b) at least one source of hypochlorite anion; and c) at least one catheter having at least one lumen. The system may further comprise at least one syringe and at least one conduit. This system may be used, for example, where the target site is a tumor, an atherosclerotic plaque, or a site of pathogenic infestation.

This invention is also directed to apparatuses for singlet oxygen delivery comprising a) a first reservoir for containing at least one peroxide source; b) a second reservoir for containing at least one hypochlorite anion source; c) a first conduit connecting the first reservoir to a delivery port; and d) a second conduit connecting the second reservoir to the delivery port. The apparatus may further comprise a mechanism to simultaneously deliver the peroxide source and the hypochlorite anion source, and/or a mechanism to control the flow of the peroxide source and the hypochlorite anion source from the first and second reservoirs through the first and second conduits to the delivery point. In apparatuses of this invention, the delivery port may be a catheter, or may be a spray nozzle, or may be any other delivery system.

The invention is further directed to apparatuses for singlet oxygen delivery comprising a) a first reservoir for containing a composition comprising at least one peroxide source; b) a second reservoir for containing a composition comprising at least one hypochlorite anion source; c) a first conduit connecting the first reservoir to a first delivery port; and d) a second conduit connecting the second reservoir to a second delivery port; wherein the first and second delivery ports are oriented to direct output to a target point. In some embodiments, the at least one peroxide source and the at least one hypochlorite anion source are solutions. As nonlimiting examples, the output may be a stream, or may be a mist. In some embodiments, the at least one of the compositions comprising at least one peroxide source or at least one hypochlorite anion source further comprises at least one surfactant.

This invention is also directed to methods for treating tumor cells or cancer cells as a result of seeding an operative site comprising administering as an irrigation or irrigating solution at least one source of peroxide and at least one source of hypochlorite anion. And the present invention is also directed to methods for killing pathogens in or on a mammal comprising administering an aqueous solution comprising at least one source of peroxide and an aqueous solution comprising at least one source of hypochlorite anion. In some methods of this invention, at least one of the aqueous solutions comprising at least one peroxide source and at least one source of hypochlorite anion further comprises at least one pharmaceutically acceptable excipient.

The invention is also directed to a singlet oxygen producing composition comprising a) at least one source of peroxide; b) at least one source of hypochlorite anion; and c) at least one of a surfactant, detergent, scent, colorant, viscosity-modifying agent, solvent, chelator, and pH-modifying agent. Methods of the invention also include disinfecting or decontaminating an inert area, comprising a) delivering at least one source of peroxide; b) delivering at least one source of hypochlorite anion; and c) delivering at least one of a surfactant, detergent, scent, colorant, viscosity-modifying agent, solvent, chelator, and pH-modifying agent. In methods of this invention, any of a), b), or c) may be performed separately, or simultaneously.

The invention is also directed to devices for combining at least two fluid reactants, comprising at least a first and a second conduit for delivering separate fluid reactants; a reaction chamber in fluid communication with said first and second conduits, wherein the reaction chamber allows for the mixing of the at least two fluid reactants; and a reaction chamber port allowing for the passage of the mixed at least two fluid reactants to the exterior of the device. Such devices include, but are not limited to, catheters, hypodermic needles, injecting-type or infiltrating catheters, spray bottles and canisters, and irrigation bottles and bags. Such devices may be gravity-driven, pressurized, or mechanically driven.

The accompanying drawings, which are incorporated in and constitute a part of this specification, illustrate several embodiments of the invention and together with description, serve to explain the principles of the invention.

### BRIEF DESCRIPTION OF THE DRAWINGS

It is to be understood that both the foregoing general description and the following detailed description are exemplary and explanatory only and are not restrictive of the invention, as claimed.

FIG. 1A diagrammatically illustrates a backpack unit in accordance with the present invention.

FIG. 1B diagrammatically illustrates how the device of FIG. 1A can be used to deliver streams of reactants to a target site

FIG. 1C diagrammatically illustrates an embodiment in which the distal ends of delivery conduits are held in place by a yoke mechanism.

FIG. 1D diagrammatically illustrates how spray nozzles produce a mist output that mixes at a target site.

FIG. 2A diagrammatically illustrates a spray bottle of the present invention.

FIG. 2B diagrammatically illustrates a spray bottle of the present invention, which includes a double trigger mechanism.

FIG. 3 diagrammatically illustrates a bottle with two chambers according to the present invention.

FIG. 4A diagrammatically illustrates a beveled-tip needle that may be used in the present invention.

FIG. 4B diagrammatically illustrates a closed-tip needle that may be used in the present invention.

FIG. 5 diagrammatically illustrates a cross-sectional view of a simple dual lumen catheter that may be used in the present invention.

FIG. 6 diagrammatically illustrates a cross-sectional view of a more complex catheter that may be used in the present invention.

FIG. 7 diagrammatically illustrates an apparatus that may be used for practicing the present invention.

FIG. 8 diagrammatically illustrates a dual lumen catheter with proximal and distal ports utilized in accordance with the present invention.

FIG. 9 diagrammatically illustrates a dual lumen catheter having a reaction chamber in accordance with the present invention.

FIG. 10A diagrammatically illustrates a hypodermic needle having a reaction chamber in accordance with the present invention.

FIG. 10B is a close-up view of the reaction chamber needle shown in FIG. 10A.

FIG. 10C diagrammatically illustrates a different embodiment of a reaction chamber needle.

FIG. 11 diagrammatically illustrates a container for delivering irrigant solutions.

FIG. 12 is a photograph of a human skin keratosis lesion approximately 1.25 cm across, prior to treatment according to this invention.

FIG. 13 is a photograph of the human skin keratosis lesion of FIG. 12 immediately after injection with 0.4 ml of 6% sodium hypochlorite.

FIG. 14 is a photograph of the human skin keratosis lesion of FIG. 12 immediately after injection with 0.4 ml of 6% sodium hypochlorite and 0.4 ml of 3% hydrogen peroxide.

FIG. 15 is a photograph of the human skin keratosis lesion of FIG. 12 three minutes after injection with 0.4 ml of 6% sodium hypochlorite and 0.4 ml of 3% hydrogen peroxide.

FIG. 16 is a photograph of the human skin keratosis lesion of FIG. 12 four hours after injection with 0.4 ml of 6% sodium hypochlorite and 0.4 ml of 3% hydrogen peroxide.

FIG. 17 is a photograph of the human skin keratosis lesion of FIG. 12 twenty-four hours after injection with 0.4 ml of 6% sodium hypochlorite and 0.4 ml of 3% hydrogen peroxide.

FIG. 18 is a photograph of the human skin keratosis lesion of FIG. 12 forty-eight hours after injection with 0.4 ml of 6% sodium hypochlorite and 0.4 ml of 3% hydrogen peroxide.

### DETAILED DESCRIPTION OF THE INVENTION

This invention relates to compositions, methods, apparatuses, and systems for singlet oxygen delivery. In particular, the present invention relates to the delivery of reactants that combine to produce singlet oxygen. These reactants include a source of peroxide and a source of hypochlorite anion. The reactants are delivered in amounts designed for the production of singlet oxygen at the site of delivery. Singlet oxygen has been well-studied, and numerous reviews of its chemistry and properties are available. One example is the review of singlet oxygen by Leonard I. Grossweiner, published at www.bio-laser.org.

### Compositions

The basic reaction between peroxide and hypochlorite is exemplified in the reaction below, in which one molecule of hydrogen peroxide is decomposed into one molecule of singlet oxygen and water.

H₂O₂+ClO⁻→¹O₂*+Cl⁻+H₂O

The present invention is not limited to hydrogen peroxide, however, and the source of the hypochlorite is also not limited.

The source of peroxide may be any source of peroxide, limited only by whether the compound is acceptable for the application. For example, some peroxide sources may be more or less desirable depending on whether the singlet oxygen is to be produced within or outside a living being. When used as an injected cancer treatment, e.g., intralesionally or intravenously, toxicity of reactants would preferably be low, whereas a higher degree of toxicity might be tolerable when the singlet oxygen is used as a decontaminating agent in cleaning up a biological or chemical exposure.

Of course, it should be noted that some compounds that are toxic in high concentrations may be pharmaceutically acceptable in lower concentrations. As a basic rule, toxicity should be balanced against the potential benefit. Again, as an example, it would be undesirable if a cancer treatment were more dangerous than the cancer itself, whereas even a low level of toxicity might be welcome in exchange for the decontamination of a deadly biological or chemical agent.

For example, as noted below, metal peroxides are useful in accordance with the present invention. However, the metal counterions for the peroxides may exhibit undesirable pharmacological effects. Thus, for animal and human use, metal peroxides may be less desirable than hydrogen peroxide. Yet when the application is on an inert surface, a metal peroxide such as calcium peroxide may be advantageous.

Thus, when viewed in the context of its desired application, the source of peroxide is essentially unlimited. Specific examples include hydrogen peroxide, urea peroxide, alkyl hydroperoxides, and metal peroxides. Examples of metal peroxides include alkali metal peroxides, such as calcium peroxide. Gel forms such as carbamide peroxide may also be used.

The particular source of peroxide may depend on the physical form in which it is to be delivered. For example, the peroxide may take the form of an aqueous solution if it will be delivered in liquid or mist form, or may take the form of a powder or crystal if it will be wetted before reacting. The possibilities are not limited and are determined only by the desired end use.

Also, the peroxide source may be a compound that itself forms peroxide. For example, superoxide, O₂⁻, is acted on by superoxide dismutase to produce peroxide. The superoxide itself may be a source for singlet oxygen, through its reaction with a hydroxyl radical, OH⁻. Superoxide may be used as its gas phase, which may be generated by microwave radiation of oxygen at 2450 Hz. This embodiment would be useful where intrapulmonary lesions or pathogens are treated by inhalation of a superoxide gas.

The source of hypochlorite anion is also limited only by what the particular end use dictates, weighing the disadvantages against the advantages. Thus, the source for hypochlorite anion is essentially unlimited as well. Hypochlorite may be provided by metal hypochlorites and/or hypochlorous acid. Metal hypochlorites include, but are not limited to, calcium hypochlorite, sodium hypochlorite, lithium hypochlorite, and potassium hypochlorite. Other sources of hypochlorite include those that may form or decompose to hypochlorite, such as, for example, chlorine dioxide.

Again, common sense dictates what compounds will be appropriate for particular applications. For example, in animal and human applications, lithium hypochlorite may exhibit unwanted pharmacologic effects, and sodium hypochlorite may be more appropriate. For inert surfaces, however, lithium hypochlorite may be the more desirable compound.

In alternative embodiments, instead of being produced from the peroxide and hypochlorite reaction, singlet oxygen may be produced from superoxide, and in particular, potassium superoxide. (See, Khan, Science 168:476-477 (1970).) In still other embodiments, singlet oxygen may be produced using radiofrequency, as described by Corey & Taylor (J. Amer. Chem. Soc. 86: 3881 (1964)).

The reactants, peroxide and hypochlorite anion, may be delivered in whatever physical form is desirable for the user. For example, nebulized, atomized, aerosolized, and in solutions, gels, solids, semi-solids, pastes, powders, mists, sprays, foams, suppositories, emulsions, lotions, douches, flushing solutions, sponges, troches, and other forms may be produced. The reactants may be delivered in sustained release form. For example, two separate solid or semi-solid implants, each with a different reactant, may be implanted in the locality of the tumor, to release their contents for a sustained reaction. As another example, a solid and liquid may be utilized; the solid form of one reactant held in place such that the liquid form of the other reactant flows over or comes in contact with the solid, thereby producing singlet oxygen. Routes of administration may be varied as well. For example, the compositions may be injected intravenously, intradermally, intraperitoneally, subcutaneously, and/or subdermally.

A single implant, divided into two solid reactant halves, may be administered. In another alternative, fluid reactants are injected, but designed to harden once in place, thereby releasing reactant over a period of time. In another alternative, separate granules of the reactants are interspersed in a single tablet or capsule, only to react upon dissolution.

The flexibility of form is also advantageous in applications outside of a living body. For example, a decontaminant foam may be prepared that includes a source of peroxide and hypochlorite in sustained release form, so that the reactants may be released over a period of time, increasing the effectiveness of the decontamination. Obviously, the choice is determined by the end use, and the disadvantages and advantages of the particular delivery route will be considered in making the choice.

Solutions have the advantage of rapid mixing, but may be more difficult to work with. Gels may not mix as quickly, but may be handled more easily. Solutions may have the disadvantage of more rapid dissipation into the surrounding area, as opposed to gels or pastes, which tend not to rapidly diffuse or dissipate. Depending on the desired result, it may be advantageous to deliver a gel with one reactant into the delivery site, followed by delivery of a liquid. The opposite may be desirable under other circumstances. Obviously, the particular combinations are left to the practitioner, and can be easily determined and then modified as necessary.

Similarly, the order of delivery is left to the practitioner. The peroxide source may be delivered first, followed by the hypochlorite anion source, or vice versa. It should be noted that because living organisms often have mechanisms, e.g., catalase or peroxidase, for destroying peroxide, it may be desirable to deliver the hypochlorite first, to avoid unwanted destruction of the peroxide reactant prior to the reaction.

The reactants may be delivered from separate reservoirs, combining only at the target site. This embodiment may be advantageous if an immediate reaction is desirable, and in such case, a solution of each reactant could be used. As it is believed that the lifetime of singlet oxygen is only 50 nanoseconds, it may be an advantage to keep reactants from reacting until in place at the target site.

In determining the appropriate dose, effectiveness is balanced against toxicity. Hydrogen peroxide has been administered to animals in the past, and published studies provide much in the way of guidance for avoiding toxic doses in internal administration. The following discussion is intended to enlighten that aspect of the invention.

One of the first reported cases of infusion of an intravenous hydrogen peroxide solution was by T. H. Oliver, in which he described a high rate of success in influenzal pneumonia. (Oliver, T. H., et al. Influenzal pneumonia: the intravenous injection of hydrogen peroxide, Lancet 1:432-433 (1920)). But it is the late Dr. Ch. H. Farr who should be credited with the more recent advancements in this area. (Farr, C. H., Rapid Recovery from Type A/Shanghai Influenza Treated with Intravenous Hydrogen Peroxide, OnLine J. of Alt. Med. Vol. 1, Bio-Oxidative Medicine Section (1993); Charles H. Farr, M.D., Ph.D., The Therapeutic Use of Intravenous Hydrogen Peroxide (Monograph), Genesis Medical Center, Oklahoma City, Okla. 73139 (January 1987); Dormandy, T. L., In Praise of Peroxidation, Lancet 11:1126 (1988)). His guidelines for preparation for intravenous peroxide solutions are as follows:

Dr. Farr begins with 30% hydrogen peroxide of USP food or cosmetic grade. Thirty percent hydrogen peroxide is a powerful oxidizer and should be handled with extreme caution.

The 30% solution is diluted with equal amounts of sterile distilled water to make a 15% stock solution. The stock solution is passed through a Millipore 0.22 μm medium flow filter for sterilization and removal of particulate matter. The stock solution is stored in 100 ml sterile containers and kept refrigerated for future use.

The infusion solutions are then prepared using sterile 5% dextrose in water. The addition of 0.25 ml of sterile 15% hydrogen peroxide stock solution to each 100 ml of carrier solution produces a 0.0375% concentration that is finally used for the intravenous infusions.

Also, it should be noted that the action of inspired oxygen with hemoglobin can produce superoxide, which when acted upon by the enzyme superoxide dismutase, yields peroxide. In this ongoing bodily process, this hydrogen peroxide is reduced by the enzyme catalase to oxygen and water. Thus, there exists a biofeedback system between catalase activity, inspired oxygen and hydrogen peroxide levels. This system helps maintain a blood level of hydrogen peroxide at 288+/−185 uM according to studies of Varma. These peroxide concentrations are helpful in determining the lower limit of solution concentrations for intravenous singlet oxygen perfusion/infusion by the present method. (For additional information, reference is made to Finney, J. W., et al., Removal of cholesterol and other lipids from experimental animals and human atheromatous arteries by dilute hydrogen peroxide, Angiology 17:223-228 (1966); Lebedev, L. V., et al., Regional oxygenation in the treatment of severe destructive forms of obliterating diseases of the extremity arteries, Vestn Khir 132:85-88 (1984)). Additional safety guidelines for hydrogen peroxide can be found on the internet at Website http://www.ee.surrey.ac.uk/ssc/h202conf/dmaftie.html.

Obviously, the concentrations of the reactants may be varied. The reactants generally are used in amounts sufficient to generate an effective amount of singlet oxygen at the target site. It has been suggested that 10¹⁰ molecules of singlet oxygen are necessary to kill a single cell. (Oseroff et al., Antibody-targeted photolysis: selective photodestruction of human T-cell leukemia cells using monoclonal antibody-chlorin e6 conjugates. PNAS U.S.A. Vol. 83(22): 8744-8748 (1986).) Clearly, the concentrations may be adjusted as needed, and one of skill in the art may determine which concentrations will be most effective for the particular application. As a nonlimiting example, the concentrations of the peroxide source may range from nanomolar to molar, e.g., from as low as 0.1 nanomolar to as high as 10 molar. Ten molar is a little higher than 30% hydrogen peroxide, and while concentrations higher than 10 M may be used, they should be used with great care due to the strong reactivity of peroxide.

The peroxide and hypochlorite may be present in equimolar amounts, and an equimolar ratio is advantageous in allowing a complete reaction. Thus, the concentration of hypochlorite may range from as low as 0.1 nanomolar to as high as 10 molar. From a practical standpoint for many applications, however, the concentration of hypochlorite, and similarly, peroxide, will be less than one molar. Exceeding this concentration for either reactant may produce unwanted, or premature, oxidation from the individual reactants alone. Of course, higher concentrations may be used, but the oxidizing effect of both reactants becomes very strong, and may be limiting. The reactants may be delivered at concentrations of approximately 10 M or less, including concentrations of approximately 2 M, 1.8 M, 1.6 M, 1.4 M, 1.2 M, 1.0 M, 0.9 M, 0.8 M, 0.7 M, 0.6 M, 0.5 M, 0.4 M, 0.3 M, 0.2 M, 0.1 M, 900 mM, 800 mM, 700 mM, 600 mM, 500 mM, 400 mM, 300 mM, 200 mM, 100 mM, or less.

The concentrations of the reactants should be adjusted to achieve the desired result. In applications where toxicity may be an issue, such as in a topical antiseptic formulation, concentrations of reactants may be decreased. But where there is little danger of an adverse oxidation effect, such as in disinfecting or decontaminating an inert area, concentrations may be increased. Also, lower concentrations may be acceptable to reduce the population of a particularly susceptible pathogen, mutated, or abnormal cells, whereas higher concentrations may be needed to oxidize a chemical agent or spore form of a pathogen.

If a greater local concentration of singlet oxygen is desired, higher concentrations of reactants will be delivered, and vice versa for lower local concentrations. Similarly, if it is determined that higher concentrations are too toxic, concentrations of one or both reactants may be decreased. Within a living body, the sensitivity to the treatment may depend on the particular application, i.e. tumor, atherosclerotic plaque, or beta amyloid deposit, the size of the area being treated, the anatomical location, and on whether the singlet oxygen is used for its vasoconstrictive effect, as well as on the local blood supply.

Volume delivered will also vary, depending on the circumstances and preferences of the practitioner. For example, the volume desired for decontamination of a room or outdoor area will obviously be much greater. Porous materials generally require a greater volume to penetrate pores and crevices, whereas smooth materials can be treated with less. Also, the volume may be increased to treat an especially contaminated area, or may be decreased if simple cleaning is desired.

Within a living body, a small tumor mass may require only 0.5 ml of total reaction volume, whereas a large mass may need 5 ml. Volume injected may be varied as desired to optimize therapeutic effects in relation to side effects and/or end result. Other factors a medical practitioner might consider in determining dose include the aggressiveness of the tumor. For example, a benign tumor might be treated with a lower concentration, or with smaller volumes. However, a very aggressive tumor might be treated with higher concentrations, or volumes that completely invade the entire tumor tissue. These choices are left to the medical practitioner. Therapy regimes are also left to the medical practitioner. For example, a practitioner may decide to repeat administrations over a period of time ranging from hours to days to weeks or months. Alternatively, a single administration may be determined to be sufficient.

An injectable composition according to the present invention may be based on lactated Ringer's solution, dextrose in water (e.g. 5%), dextrose in normal saline, or ethanol, for example. Topical formulations may be based on a solvent, such as, for example, ethanol or dimethyl sulfoxide. Other topical formulations may be based on glycerin, aloe, lanolin, etc.

The formulations may contain other ingredients, depending on the desired use. For example, a decontaminating foam may include detergents or surfactants, or other agents that enhance the foaming effect. Other ingredients may be added for other purposes, and the composition may include surfactants, detergents, scents, colorants, viscosity-modifying agents, solvents, chelators, and pH-modifying agents. The choice of additional elements in the composition is the choice of the practitioner.

In some instances, a single administration of the composition of the invention may be sufficient to achieve a positive result. In other instances, repeated administration may be necessary. The frequency and concentration of administration will depend upon the results obtained, and may be modified as necessary. For example, where the application is a decontamination effort, samples or cultures should be taken from the contaminated area after treatments to ascertain the level of success.

Indeed, where administration is directed to inert, inorganic, or even organic surfaces, Gram staining, microscopic examination, biochemical and enzymatic tests, carbohydrate fermentation reactions, and/or gas chromatography of metabolic fermentation products, could be used to ascertain the effectiveness of treatment. These methods may be used, in particular, in testing with swabs or cultures from surfaces or aspirates, abscesses, etc.

Where the administration is to living tissues, the dosing will be determined by the response observed. For example, a single administration may be sufficient to obtain significant necrosis in the treated area. After approximately one week, the treated area should be observed to determine whether, and to what extent, treatment should be repeated. The treatment site may be “observed” by use of radiographic or endoscopic techniques, for example. A decision to treat again may be based on a reduction in the size of the treated lesion.

For external administration, observation is more easily accomplished. Necrosis should be visible in the treated area by three or four days after treatment, and a repeat administration may be useful at that time.

Other details of applications and methods of delivery will be presented below in greater detail.

### Applications

Because of its potent oxidizing potential, singlet oxygen is useful in a number of applications in accordance with the invention. For example, following the Sep. 11, 2001, terrorist attacks, there has been a global wakeup call to find ways to counter and/or control biological and chemical warfare agents. The present invention is ideally suited for that purpose.

Biological agents that are of great concern from a biological warfare standpoint are anthrax, brucellosis, botulism, cholera, plague, and small pox. Typical chemical agents include sarin, tabun, VX, soman, cyanide, and mustard/blistering agents. The task of securing an area of attack and of ascertaining the nature and severity of a toxic threat is usually given to the first responders, which consist of fire fighters, police officers, emergency medical personnel, and military personnel. Usually, a thorough search of the area must be a priority at the onset of an attack and in the case of a biological/chemical warfare incident, a large down-wind area must be secured and/or evacuated. And once an attack has been made, decontamination and containment becomes a problem.

Because the agents used in biological or chemical attacks are capable of being oxidized, the present invention is useful in their decontamination. In one embodiment, separate aqueous reservoirs of hydrogen peroxide and sodium hypochlorite are prepared, and the reactants are combined at the site of contamination. Pouring or spraying the separate reactants onto the affected area, either sequentially or simultaneously, is one manner in which the area may be treated. The reactants may be applied to large areas from the air with tanker planes, bucket-type helicopters, or crop dusters. A more local application may be obtained using fire trucks, street washing machines, or other similar devices filled with aqueous solutions of hydrogen peroxide and sodium hypochlorite to produce singlet oxygen which could be used to directly saturate the affected premises.

Even more targeted application to a contaminated area may be achieved through the use of individual backpack canisters to be worn by decontamination personnel. The backpack canisters contain separate reservoirs of peroxide and hypochlorite solutions, under pressure, which are delivered to the target area by the decontamination personnel. Canisters such as those mentioned here, and other delivery devices, are detailed below.

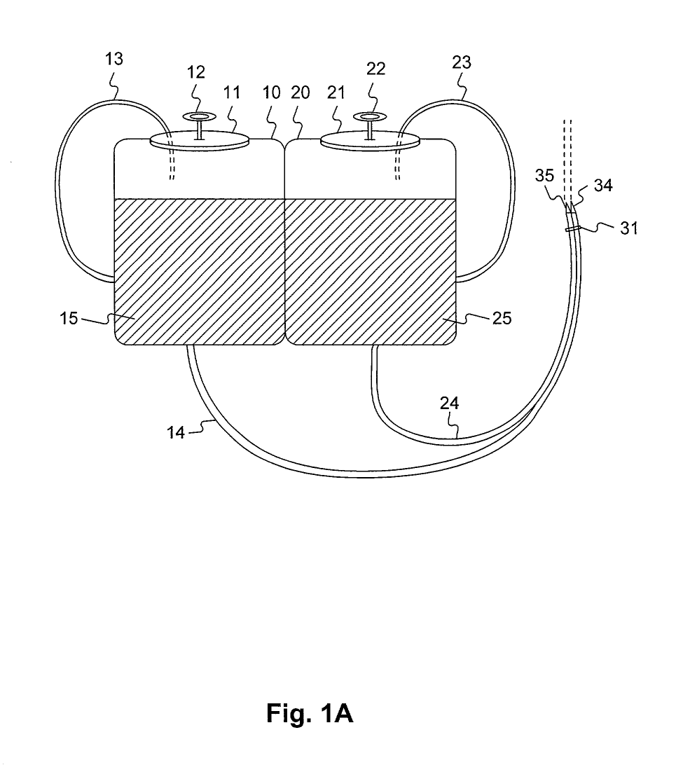

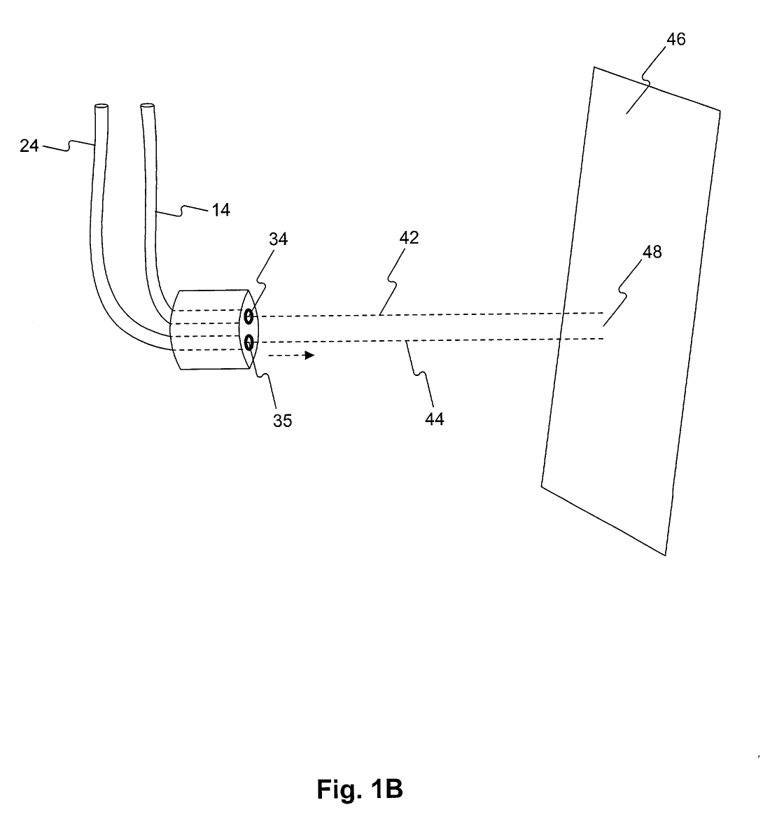

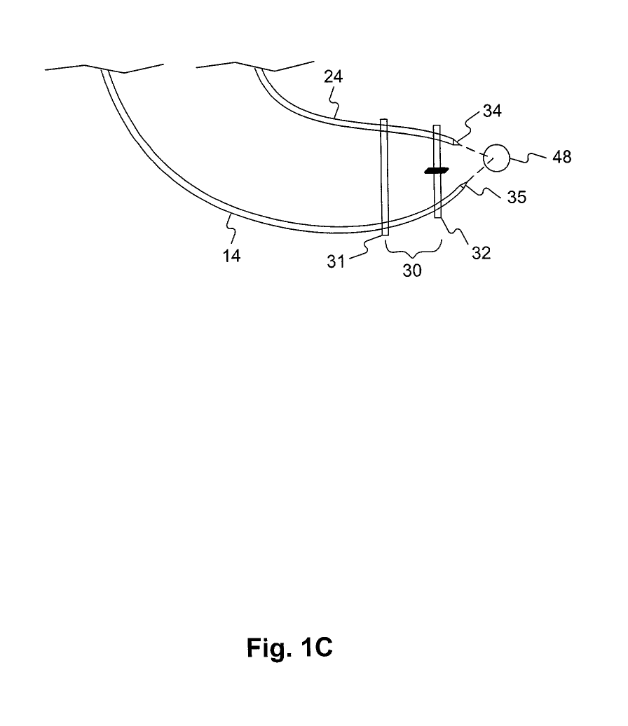

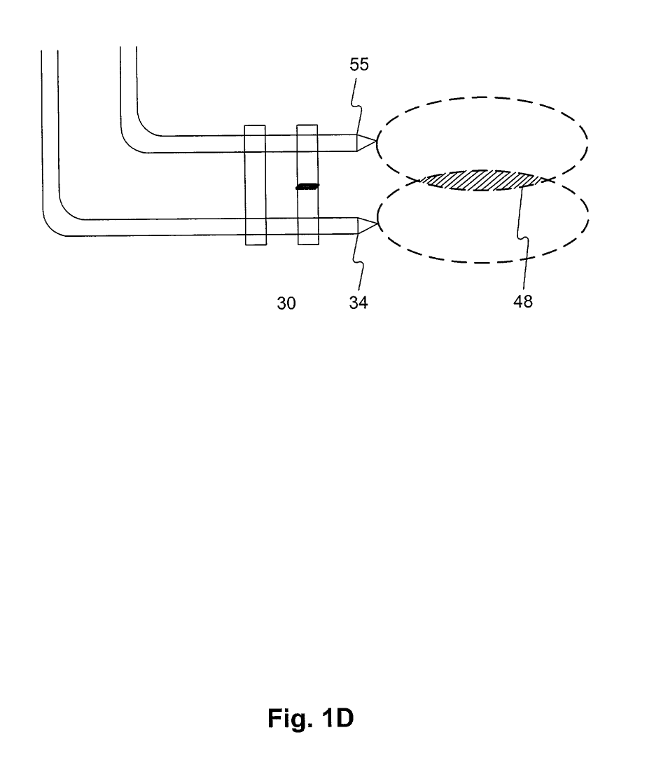

A pressurized backpack unit is shown generally in FIG. 1A. The backpack unit includes two canisters 10 and 20. In other embodiments a single divided canister serves the same purpose. Each canister includes a screw top, 11 and 21, for pouring the reactants, 15 and 25, into the respective canisters. A pump in each canister, 12 and 22, is used to introduce air into the canister to pressurize the contents. Shoulder straps, 13 and 23, secure the canister to the decontamination personnel. Separate delivery conduits, 14 and 24, deliver the pressurized contents to the target site through spray nozzles 34 and 35. In alternative embodiments, the canister is not pressurized and delivers its contents by the force of gravity. In other alternative embodiments, a reaction chamber combines the reactants prior to delivery.

In the embodiment shown in FIG. 1A the delivery conduits are joined together to deliver their respective contents in parallel to the target site, mixing on contact. A trigger mechanism 31 controls the output from the conduits. FIG. 1B shows a diagrammatic view of how the device of FIG. 1A delivers a parallel stream of reactants to a target site. Delivery conduits 14 and 24 deliver reactants through spray nozzles 34 and 35 to deliver streams of reactants 42 and 44 to a surface 46 where a reaction takes place at target site 48.

FIG. 1C shows an alternative embodiment in which the distal ends of the delivery conduits 14 and 24 are held in place by a yoke mechanism 30. The yoke includes a trigger 31 and an aiming harness 32, which is used to angle the output stream through spray nozzles 34 and 35 to a target site 48 for mixing. FIG. 1D shows a different embodiment in which the spray nozzles, 34 and 35, produce a mist output that mixes at target site 48.

In other embodiments, high pressure washing machines, generating pressures of up to 3500 psi, are used. Washer nozzles producing a fan-shaped spray, such as a 14-degree washer nozzle, may be used. Sprays or mists may be directed so as to converge at a target site some distance from the washer nozzle, such as from approximately 5 to 15 feet beyond the nozzle.

The present invention may be used where there is a need for a rapid response deployment unit located in a suspected target area. The active reagents of peroxide and sodium hypochlorite (in appropriate concentrations) can be readily and safely stored in, for example, military installations. This would give a wide distribution of these potentially life-saving reagents and since they are stable when properly stored, would make them readily available. Because these reagents are easily and economically produced, this method provides for comprehensive emergency protection from both biological and chemical warfare agents.

This application is especially desirable as compared to existing cleanup methods because: 1) the reactants are readily available; 2) the reactants are relatively inexpensive; 3) the reactants are stable in storage; 4) the reactants can be totally miscible with water, resulting in easy cleanup; 5) the reactants are quickly manufactured for additional supplies and/or in large quantities; 6) the reactants and products are primarily nontoxic in concentrations to be used; 7) excess or residual reactants are broken down by auto oxidation, ultraviolet light, and sunlight; 8) singlet oxygen is highly effective as a decontaminating agent; and 9) the reactants and product are non-mutagenic.

The present invention is also particularly applicable for use in public or industrial works. For example, where large volumes or liquids are stored, passed, or carried, growth of unwanted microorganisms, including Legionella, or even amoebal, algal, or protozoal growth, can be problematic. Specific examples include water in cooling towers, pipes, water supplies for municipalities, swimming pools, and other large stores of water, where microorganisms have a place to thrive. Other examples include main water supplies, vegetable wash water, meat process water, pasteurizers, water recycling, effluent treatment, spiral spin chillers, irrigation water, and hydroponic feed water. The potent oxidizing effect of singlet oxygen makes this invention especially useful in preventing and treating such microorganisms.

These same advantages make the present invention useful in more common applications, such as in the sterilization of hospital settings, especially operating rooms and surgical instruments, in the disinfection of bathroom floors, sinks, toilets, and tubs, and in the general cleaning of other areas in which a disinfectant or sterilant affect is desired. For example, a squirt bottle with a septum can be used to hold and simultaneously deliver aqueous solutions of peroxide and hypochlorite to a site at which singlet oxygen would be produced. A spray bottle or canister with two reservoirs could be used in this manner as well, for cleaning up restaurant or kitchen countertops and tables.

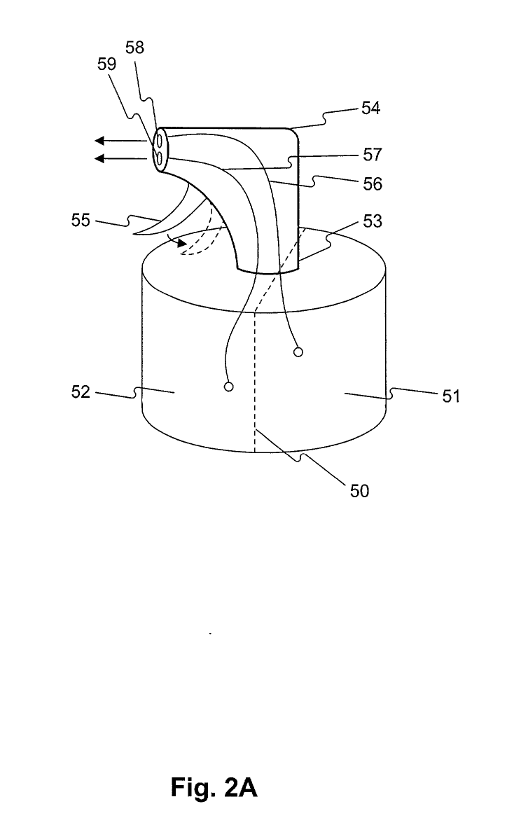

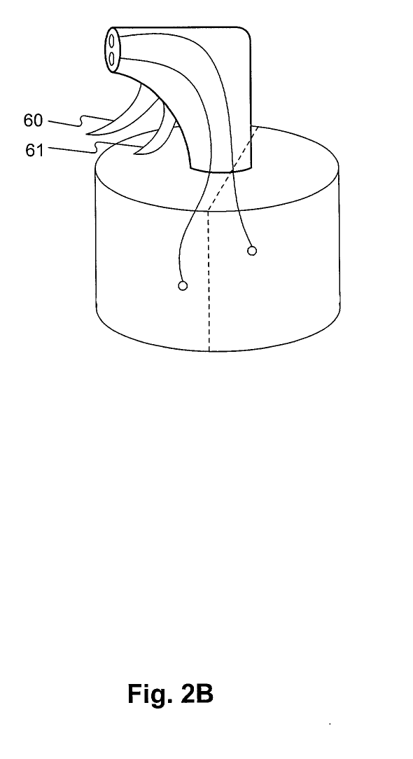

FIG. 2A diagrammatically illustrates a mechanically driven spray bottle of the present invention. In the embodiment shown, the separate peroxide and hypochlorite anion sources are kept in separate compartments, 51 and 52, of the spray bottle. A septum 50 divides one compartment from the other. A single screw top opening straddles the two compartments and a spray nozzle 54 is attached. The spray nozzle 54 includes a trigger 55. Actuating the trigger initially pulls and delivers a precise volume of a first reactant from compartment 51 through delivery conduit 56. Continued actuation of the trigger pulls an equal volume of the second reactant from compartment 52 through delivery conduit 57. In this manner, a single actuation of the trigger consecutively delivers a first and then second reactant through separate delivery ports, 58 and 59, of the nozzle. The reactants combine at the target site to produce singlet oxygen.

FIG. 2B shows an alternative trigger embodiment, which includes a double trigger mechanism. Trigger 60 pulls and delivers a precise volume of a first reactant from compartment 51 through delivery conduit 56. Actuation of trigger 61 pulls an equal volume of the second reactant from compartment 52 through delivery conduit 57. In this manner, actuating the double trigger mechanism sequentially delivers a first and then second reactant through separate delivery ports, 58 and 59, of the nozzle. The reactants combine at the target site to produce singlet oxygen.

Because of the nontoxic nature of the reactants and products of this invention, at an appropriate concentration, compositions of this invention may be applied directly to the skin for an antiseptic effect. For example, aqueous solutions of hydrogen peroxide and sodium hypochlorite are delivered as a mist from a spray bottle onto an area of the skin being prepared for surgery. In this embodiment, a single trigger mechanism would simultaneously deliver a mist of both reactants at the target site. In this manner, the fine mist contacts and saturates the surface area of the skin, greatly reducing the population of pathogens by the oxidizing effect of singlet oxygen.

In another embodiment, the separate reactants are applied separately. For example, separate spray or squirt bottles of peroxide and hypochlorite are made available for cleansing an area of skin to be treated. Alternatively, sponges may be used to apply the reactants, or the reactants may be supplied in pre-packaged individual “prep pads,” which are saturated in either peroxide or hypochlorite. The desired effect in these embodiments is to rid the skin of unwanted pathogens.

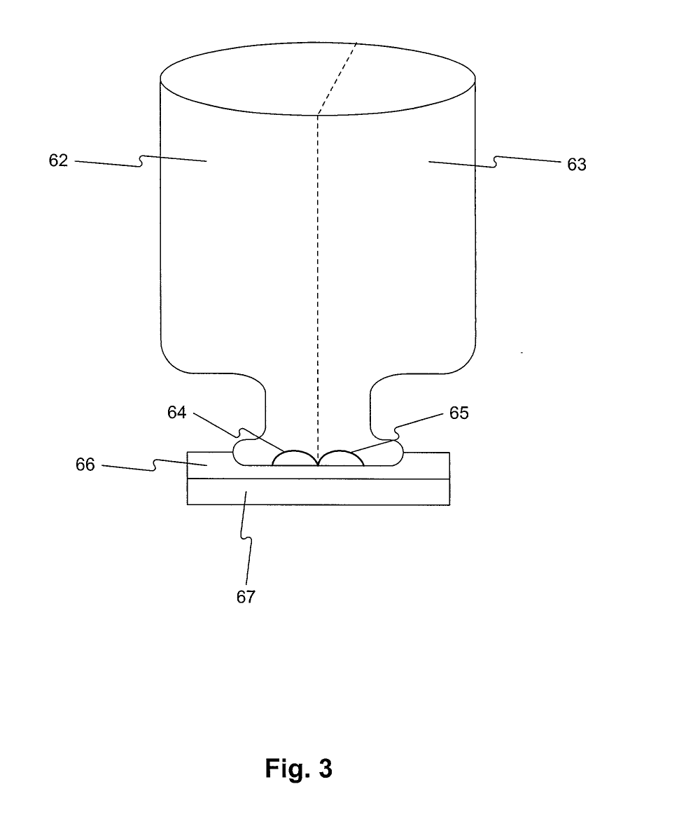

In other embodiments, the invention also finds use in topical applications as an effective exfoliant for the skin. As an exfoliant, the invention may be used to treat precancerous and cancerous skin lesions. The reactants may be supplied in two separate topical application bottles, to be applied sequentially, or in a single bottle with two chambers so the reactants are mixed during application to the skin. An example of such a bottle is illustrated in FIG. 3.

The bottle of FIG. 3 includes two chambers, 62 and 63, to contain the separate reactants. The bottle is designed to deliver by gravity or pressure the contents of the two chambers through delivery ports 64 and 65, respectively. The bottle is designed to deliver the reactants in equivalent volumes. An absorbent pad 67 is held against the delivery ports by a track 66. When the bottle is inverted or squeezed, reactants from the separate chambers are delivered simultaneously into the absorbent pad, which may then be applied topically to an area to be treated. In the embodiment shown, the used pad 67 may be removed after use, and replaced with a new pad for a new use. In alternative embodiments, the bottle is designed for a single use and the pad is made integral to the bottle.

The nontoxic nature of the reactants and products makes the present invention applicable in numerous applications. This nontoxic quality is especially important in applications in which the reactants are introduced into a living body to produce a reaction within. As nonlimiting examples, cancer, atherosclerotic plaques, or even dental plaques, may be treated in accordance with this invention. In the case of the tumor, the oxidizing effect of singlet oxygen is used to destroy cancer cells, and in atherosclerosis, the singlet oxygen oxidizes components of the plaque.

Because the reactants are consumed in the reaction, a highly localized effect is produced. Thus, the invention is useful in local killing of cells, where more widespread destruction is undesirable, and targets for the singlet oxygen therapy include, for example, lesions, tumors, and cancer. Target sites range from the benign wart, keratoses, papillomas, to benign tumors, and even to malignant cancer.

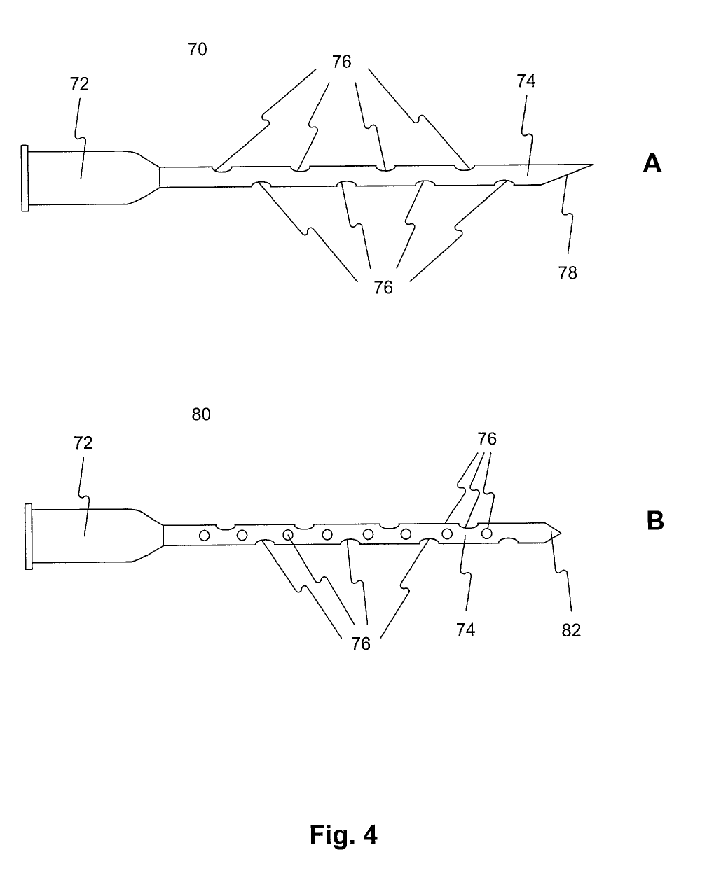

The means for delivery of the reactants to the target site may be designed to deliver the reactants sequentially or simultaneously. For sequential delivery, two syringes with needles to penetrate to the depth of the target are all that is needed. An anesthetic may be used to desensitize the area prior to treatment. The needle for delivering the reactants to the target site may be a conventional hypodermic needle, or may be a perforated hypodermic needle, as shown in FIG. 4.

Examples of perforated hypodermic needles that may be used in accordance with the present invention include the needles of FIG. 4A, generally shown as 70 and 80. These needles include a plastic Luer-locking base 72 for attachment to a syringe. A stainless steel shaft 74 includes perforations 76 for allowing injected materials to be ejected radially from the needle. Embodiment 4A includes a beveled tip 78, whereas embodiment 4B includes a closed tip 82.

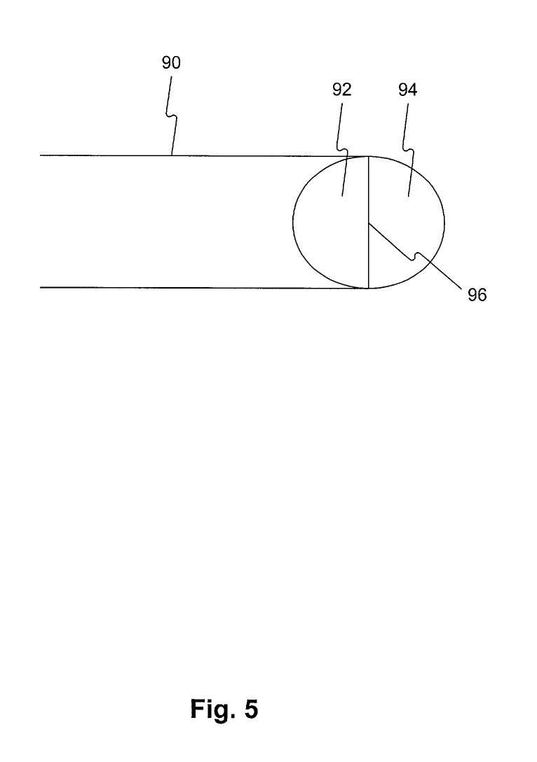

For simultaneous delivery, a dual lumen catheter may be used. FIG. 5 diagrammatically illustrates a cross-sectional view of a very simple dual lumen catheter 90 that may be used in the present invention. Catheter 90 includes a first lumen 92 for delivery of the first reactant and a second lumen 94 for delivery of the second reactant. The lumens are separated by a septum 96. Other dual-lumen catheters, such as those formed with concentric lumens may also be used.

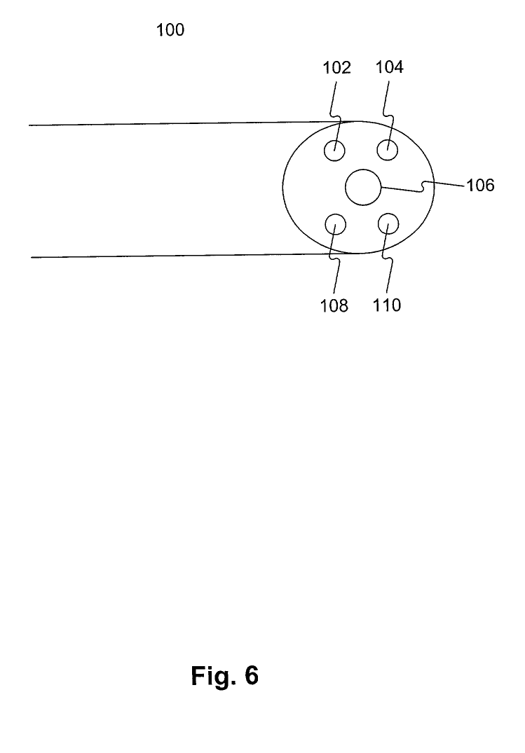

Still more complicated catheters may be designed or used, for example, where there is a need for an endoscope for optical guidance to the treatment site. Thus, the catheter capable of delivering two reactants may be endoscopically guided to the tumor site, where the reactants are simultaneously (or sequentially) injected. An example of such a catheter is shown in FIG. 6. A catheter might also be guided to a target site using standard radioscopic or endoscopic techniques. For example, radio opaque catheters may be placed using a guide wire and monitored using x-ray technique. This would be advantageous for lesions in the peritoneum, gut, stomach, bronchus, thoracic cavity, etc.

The catheter of FIG. 6, generally 100, includes a first lumen 102 for delivery of a first reactant and optionally the sequential delivery of a second reactant, and an optional second lumen 104 for delivery of a second reactant. A lumen 106 for an endoscope may be placed generally in the center of the catheter. Lumens for electro-cautery 108 and suction or vacuum 110, both optional, are also shown in the Figure. In other embodiments, different combinations of lumens are provided for different applications. For example, a lumen may be used for an endoscopic camera. Other applications are within the scope of the invention.

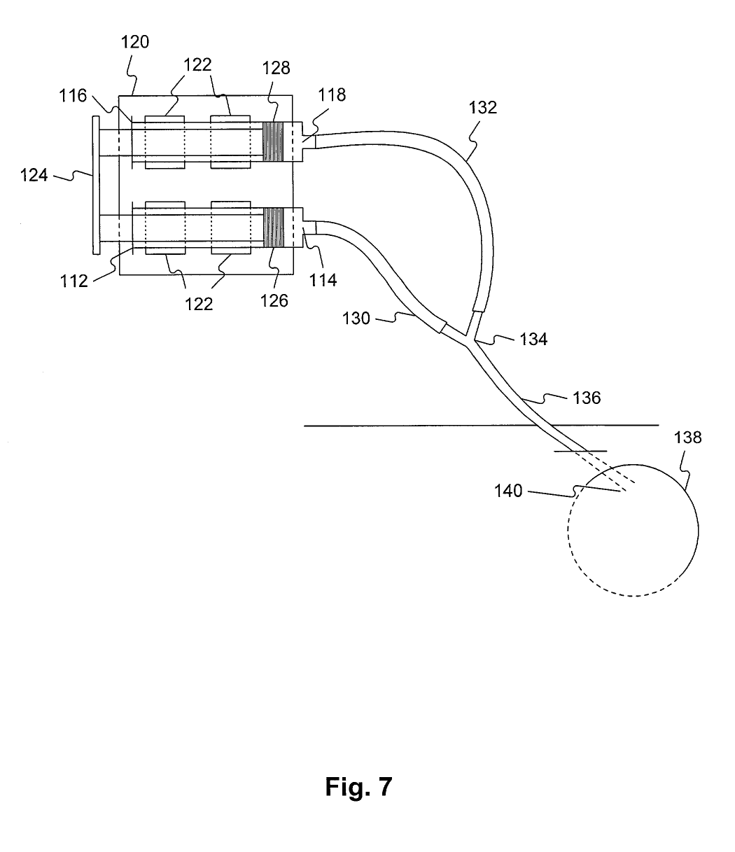

FIG. 7 diagrammatically illustrates one embodiment of the present invention in use. The system shown in FIG. 7 includes a first syringe 112 for delivering a first reactant 114 and a second syringe 116 for delivering a second reactant 118. The syringes are mounted to a support plate 120 by brackets 122. An optional yoke 124 actuates first syringe plunger 126 and second syringe plunger 128 simultaneously. Upon actuation, first reactant 114 is forced into conduit 130, and second reactant 118 is forced into conduit 132. A Yjoint 134 of dual lumen catheter 136 brings together first reactant 114 and second reactant 118, without mixing. Catheter 136 is targeted into tumor 138. The reactants 114 and 118 only mix upon exit from the catheter at mixing point 140.

Although this particular embodiment has been described generally with reference to tumor treatment, the targeted delivery of the present invention provides for treatment of a wide array of conditions, including bacterial, fungicidal, viral and protozoal infections, infestations and other abnormal growths and deposits (including, for example, metastases, arterio- and atherosclerotic plaques, atheroma, arterio-venous malformations, amyloid deposits, dental plaques, HIV infection, systemic fungal infection, etc.), and provides for an extremely potent vasoconstrictive effect.

In another embodiment, advantage is taken of the natural fluid flow of the body to deliver reactants to the desired site. For example, the guided multi-lumen catheter is generally placed at or near the desired site or region of an infection, infestation and/or abnormal growth, and is located such that the natural direction of blood flow, whether arterial or venous or lymphatic, carries the reactants or generated singlet oxygen to the desired treatment site.

This embodiment capitalizes on the fact that the two reagents (such as hydrogen peroxide and sodium hypochlorite) are not allowed to mix or interact prior to being released at the targeted therapeutic area. With the multi-lumen catheters of the present invention, with axially spaced ports and individually separated lumens, it is possible to deliver two or more reagents to the therapeutic target and allow them to be released from different ports. The body's natural flow of arterial or venous blood will then mix the reagents such that singlet oxygen is generated and carried to and throughout the therapeutic target.

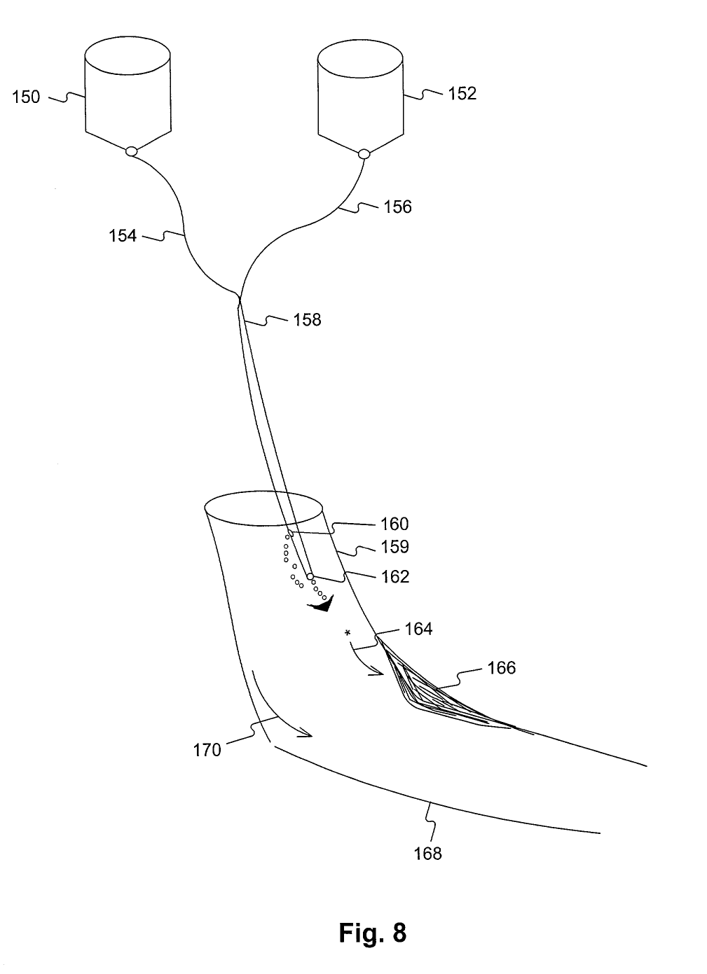

In one particular embodiment, illustrated in FIG. 8, a dual lumen catheter with proximal and distal ports is utilized. In this embodiment, separate reactant solutions are held in IV bags 150 and 152. Delivery conduits 154 and 156 carry the reactant solutions to a dual lumen catheter 158. The tip of the guided dual lumen catheter system, shown in the Figure generally as 159, is located at or near its desired treatment location within the vascular system, illustrated diagrammatically as 168. Blood flow is in the direction indicated by the arrow 170.

The tip of the dual lumen catheter 158 has a proximal port 160 from which the first reactant from bag 150 is constantly delivered. A distal port 162 located down the fluid flow 170 from the proximal port 160 constantly delivers the second reactant from bag 152. The reaction between the two reactants takes place to create a constant supply of singlet oxygen at the target site 164. In the embodiment shown, the condition to be treated is an atherosclerotic plaque 166. In alternative embodiments, this procedure may be used to treat other plaques, such as beta amyloid plaques in Alzheimer's disease.

In this model, one reactant is released from the most distal port and the other reactant from a more proximal port. This allows the first reactant to be carried by the bloodstream and mixed with the second reactant as it exits from the tip of the catheter. Consequently, singlet oxygen is perfused, infused, infiltrated or flushed through the organ or region for therapy. Since concentrations of hydrogen peroxide and sodium hypochlorite may purposely have to be kept low, the guided multi-lumen catheter can be attached to bags or bottles of these perfusate reagents and generated over time periods ranging from minutes to days of perhaps even perpetually.

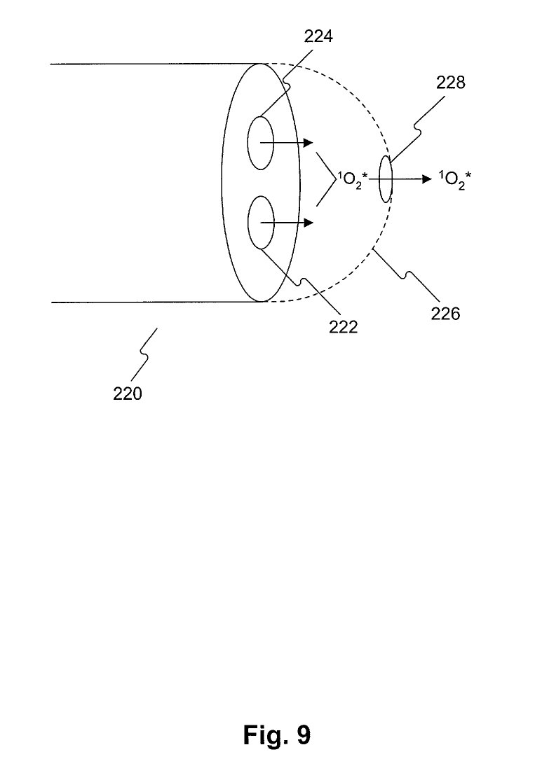

FIG. 9 diagrammatically illustrates the tip of another catheter design for use in the present invention. The catheter, shown generally as 220, is a dual-lumen type having lumens 222 and 224. An enclosed reaction chamber 226 serves as a mixing reservoir in which the reactants can react without dissipating into the surrounding blood flow. A reaction chamber port 228 serves as a point from which singlet oxygen is delivered. This design is advantageous in that it provides a reservoir in which the reactants remain at the desired concentrations, and in which the reactants are protected from breakdown by the body.

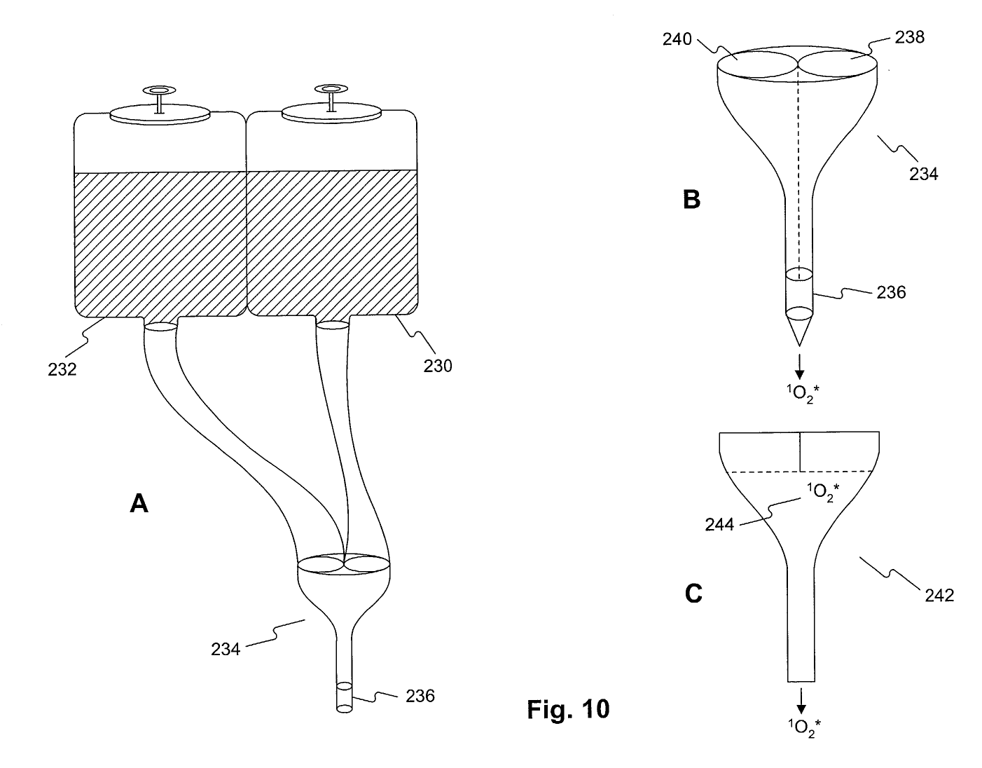

FIG. 10 diagrammatically illustrates a hypodermic needle having a reaction chamber. In the first embodiment, shown in connection with reactant reservoirs in FIG. 10A, the needle 234 is fed by separate reactant reservoirs 230 and 232. The needle prevents mixing of the reactants until reaching the reaction chamber 236. Upon reaching the reaction chamber 236, the reactants react, and singlet oxygen is produced and ejected from the needle. FIG. 10B shows a close-up view of the reaction chamber needle with separate channels 238 and 240 for keeping reactants separate. The reactants combine in the reaction chamber 236 to produce singlet oxygen.

In a second embodiment of the reaction chamber needle, shown diagrammatically in FIG. 10C, the needle 242 is much smaller. Because of the significantly reduced size, it is unnecessary to have separate channels, and the reactants flow into a central reaction chamber 244.

Devices having characteristics of both a needle and a catheter are also envisioned. For example, injecting-type or infiltrating catheters, having a sharp tip for passing through tissue, are also envisioned. These injecting-type or infiltrating catheters generally have a reaction chamber located proximally to the sharp tip. Alternatively, the reaction chamber may be formed by a sheath that is pushed over the sharp tip after the catheter has been advanced into position. In other embodiments, the sharp tip is retractable, or the sheath may protect the tip during advancement or placement.

The fact that the catheter can be guided utilizes present day well known techniques to reach a wide range of body organs, systems, regions or locations. The design of the multi-lumen catheter makes it an ideal conduit to carry the two individual reactants (such as hydrogen peroxide and sodium hypochlorite) separately without mixing before arrival at the desired therapeutic site, area or region of the body.

With regard to the reaction chamber, any device that is used to deliver at least two reactants, where the reactants are to be combined before ejection from the device, may include a reaction chamber. For example, a hand-held sprayer may include a reaction chamber in its nozzle to combine reactants prior to ejection. Similarly, a backpack canister may include two delivery conduits that join together to form a nozzle that includes a reaction chamber. In other embodiments, such as in an irrigation bottle or bag, a reaction chamber is used to mix the reactants after their delivery from their separate compartments but prior to contact of the reaction mixture with the target area.

The reaction chamber could alternatively contain a solid or semi-solid form of one or both of the reactants, wherein a liquid flows over the solid or semi-solids resulting in a solution of both reactants, which then react. The liquid itself may include a reactant as well.

Returning to the discussion of catheters and other injection devices, since both peroxide and hypochlorite (and any of their chemically active derivatives or analogs) are totally miscible in water, their respective concentrations can be elegantly controlled to achieve both therapeutic levels of singlet oxygen and to keep excess singlet oxygen to a minimum. Moreover, the body possesses the necessary enzymatic systems to deal with limited excess levels of both of these physiological agents (hydrogen peroxide and sodium hypochlorite) and converts them into carbon dioxide, water, sodium chloride, and ground state oxygen. This embodiment is useful in treating organs such as the lungs, pancreas, liver, intestine, heart, stomach, brain, etc. When reagent concentrations are kept at levels to avoid air embolism, singlet oxygen can safely be delivered to these areas as needed for therapeutic purposes.

Catheter length, diameter, design, etc., are determined by the regional anatomy and vasculature as regards the specific therapeutic application. Because of the short half-life of metastable singlet oxygen, this method of delivery utilizes its known activity to a great advantage. This method allows accurate and controlled delivery of singlet oxygen to a vast array of potential therapeutic sites. This makes its application essentially limitless.

Another advantage of this method is the treatment of not only a tumorous or cancerous lesion but also an entire area or region of its metastasis. This concept also extends to an infected area or even to septicemia or intravascular disseminated infections or infestations. This embodiment can be used to cleanse the blood in vivo. It allows guidance of treatment to sites of heavy growth of pathogenic organisms (bacterial, fungal, viral, including HIV, and/or protozoan) or tumorous lesions and produces elegantly controlled amounts of singlet oxygen in a safe, economical and reliable manner. Additionally, this embodiment can be an adjunct to or supplement to direct needle infiltration of singlet oxygen to desired therapeutic sites as described herein.

Because all of the body's blood circulates on a regular periodic basis, the blood will pass a common point such as the superior vena cava, the pulmonary artery, the right atrium, etc. By introducing the compositions of the present invention at that point, the entirety of the blood in the body may be exposed to the cleansing effects of the singlet oxygen. Alternatively, blood may be circulated extracorporeally, such as in dialysis, and exposed to the cleansing effects of singlet oxygen outside of the body. In this embodiment, the body's exposure to singlet oxygen is minimized, due in large part to the short half-life of the species.

In other instances where the effect on the body is to be reduced, lower concentrations and slower infusion rates may be used. Additionally, the body has mechanisms for breaking down hydrogen peroxide, and is able to utilize some hypochlorite. In any case, the short half-life of singlet oxygen is the ideal limiting factor to prevent undue toxic build-up.

The invention may also be used as a surgical or wound irrigant. Treatment may be performed at the excision site of a tumor or abscess, or in the thoracic, peritoneal, or cranial spaces. The singlet oxygen irrigation solution would produce its beneficial bactericidal, viracidal, and tumoricidal effects as an irrigant. In such embodiments, the reactants may be provided in a single use bottle with two separate compartments. When the bottle is opened, both reactants may be discharged into the site simultaneously. Alternatively, the reactants may be supplied in separate bottles to be used together or sequentially.

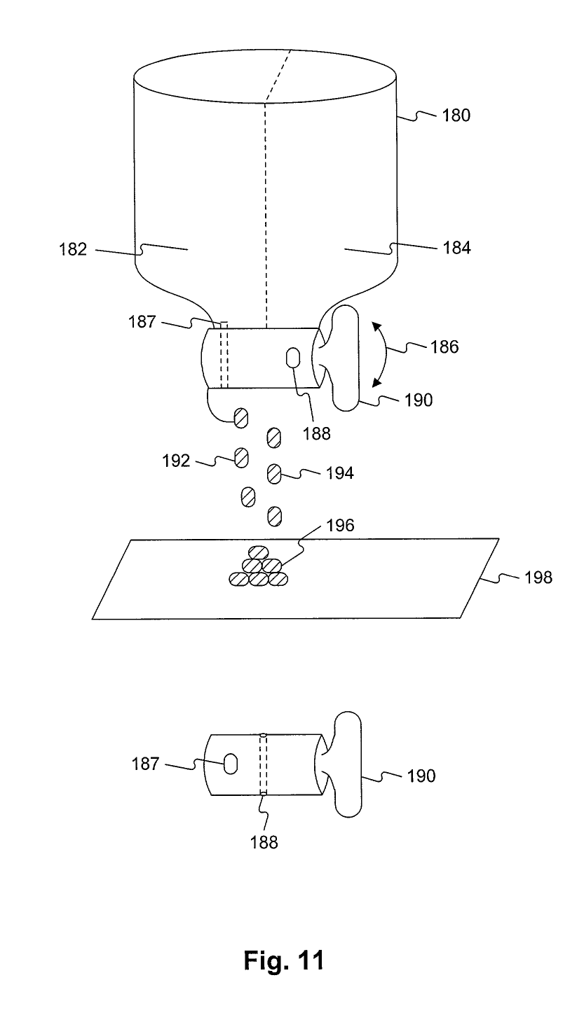

FIG. 11 illustrates a container, generally 180, which is useful in delivering irrigant solutions. Separate reactants are held in compartments 182 and 184. A stopcock 186 holds the reactants in place, and has perpendicularly placed ports 187 and 188 for alternately delivering the contents of 182 and 184. A stopcock handle 190 is turned to allow delivery of the separate reactants as drops 192 and 194, or streams, to the target site 196 of a surface 198, which may be living or inanimate. The stopcock 186 rotated by 90° is illustrated in the bottom frame of the Figure. In this embodiment, the reactants can be delivered separately without concern for premature reaction, as the stopcock is designed to separately deliver the reactants. The reactants may be delivered, for example, by pressure or gravity.

The invention is also useful as avasoconstrictor, or in applications in which blood flow to the area is to be reduced. Hemostasis is of prime importance at an incision or operative site. The invention shows the surprising effect that singlet oxygen causes intense vasoconstriction of normal blood vessels but that the effect is later cleared from the site without harmful side effects. This effect is nearly instantaneous after administration, or at the least, occurs much more rapidly than other known vasoconstrictive methods. By applying reactants in accordance with the present invention, local blood flow may be reduced. Treatment is performed in a manner similar to that in which xylocaine and epinephrine are delivered for a vasoconstrictive effect. The invention may, therefore, find use in reducing bleeding at a surgical site or wound, or in any other situation where local vasoconstriction is desired. In addition, the invention may also include combinations with, or following, topical and local anesthetic application. Such anesthetics include but are not limited to xylocaine, novocaine, pontocaine, mepivacaine, and cocaine.

Because of the nature of the reaction, including its completeness and consumption of the reactants, this invention may be repeatedly delivered or administered without undue effects. There is no long-term or cumulative accumulation of reactants or products of the reaction. Thus, this invention is ideal where repeated administration is desirable.

The following examples are intended to illustrate the invention. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result.

### EXAMPLES

### Example 1

### Treatment of Keratosis

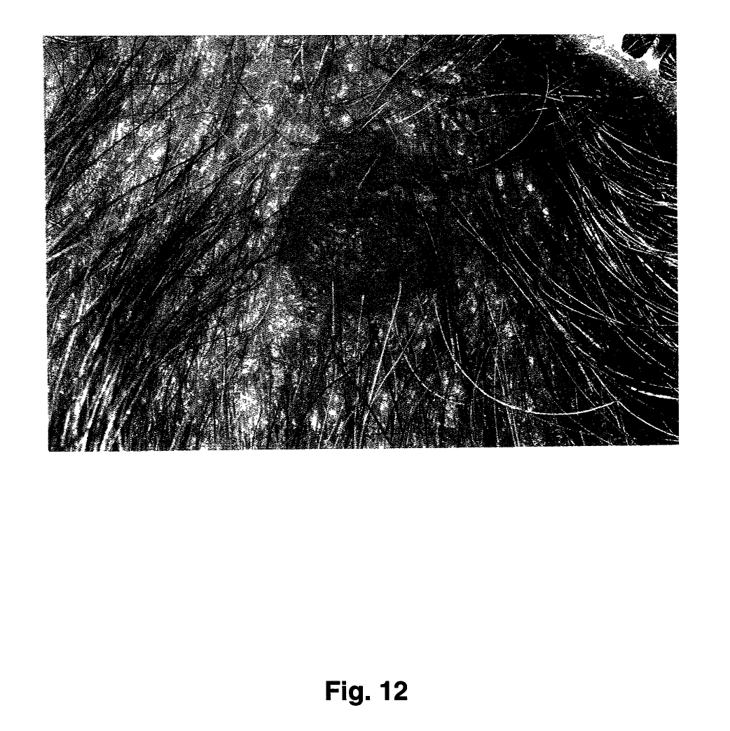

A skin keratosis lesion measuring approximately 1.25 cm in diameter was chosen as a target for treatment. The lesion was located on the left temple of a 57-year old white male. A photo of the keratosis lesion, prior to treatment, is shown in FIG. 12.

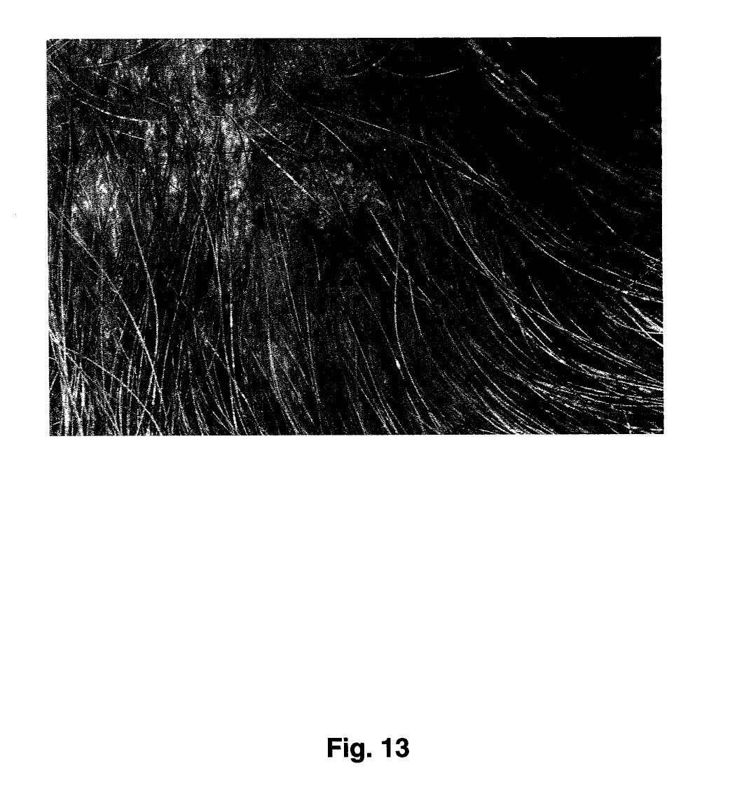

Using a 30-gauge hypodermic needle, 0.4 ml of a 6% solution of sodium hypochlorite was injected into the center of the lesion. The injection resulted in a mild burning sensation, and produced minor bleeding at the lower border of the injection site. See FIG. 13 for a photo of the area immediately following the injection.

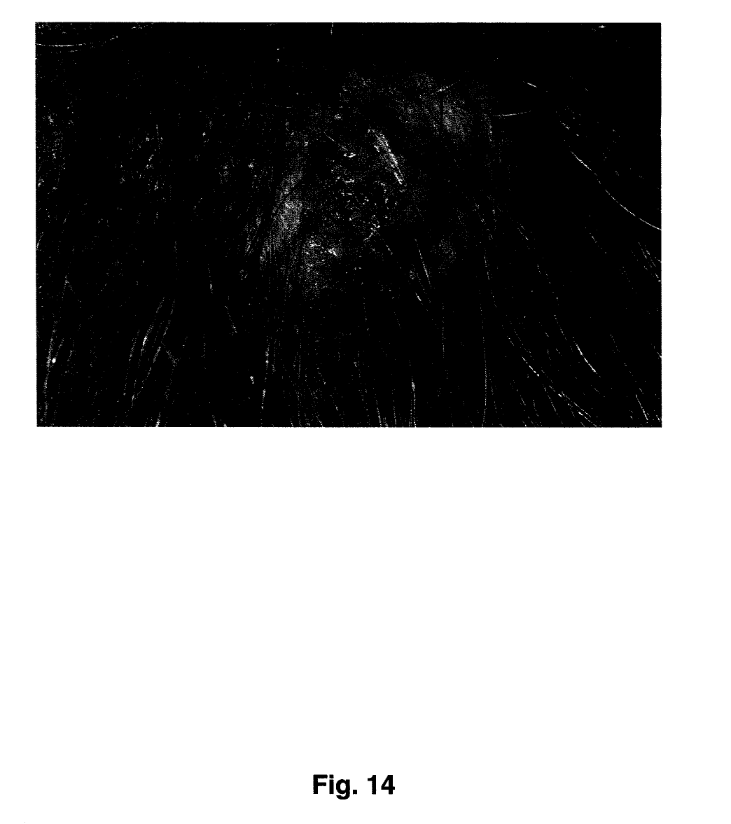

Immediately after the first injection, using a 30-gauge hypodermic needle, 0.4 ml of a 3% solution of hydrogen peroxide was injected into the center of the lesion. The injection produced foaming, or bubbling, at the surface of the lesion, and blanching of the surrounding tissue. See FIG. 14 for a photo of the area immediately following the injection.

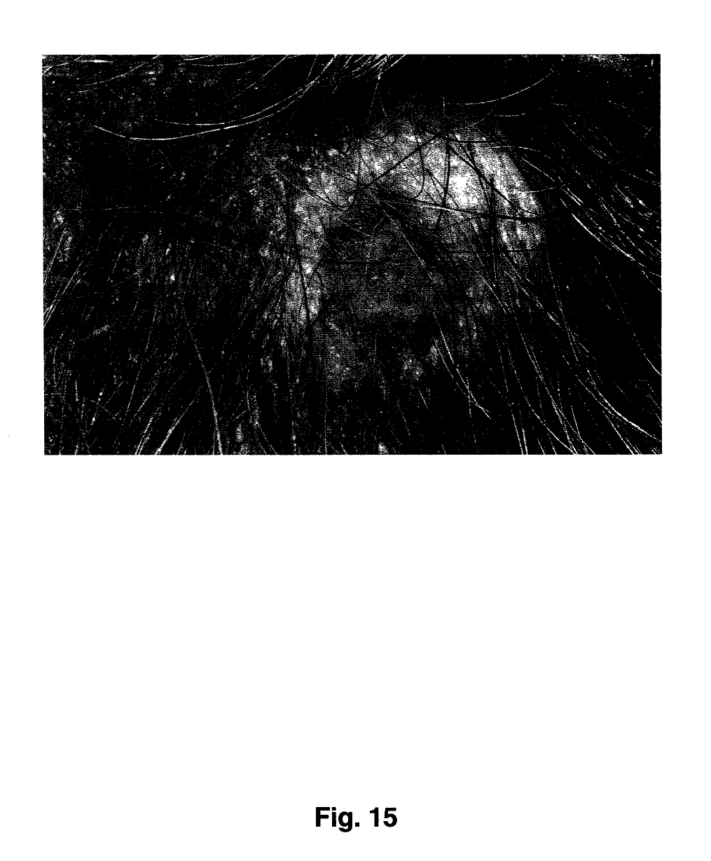

FIG. 15 shows the lesion site three minutes after injection. The marked blanching in the area surrounding the injection indicated extreme vasoconstriction. The blanched tissue was “normal” tissue surrounding the lesion.

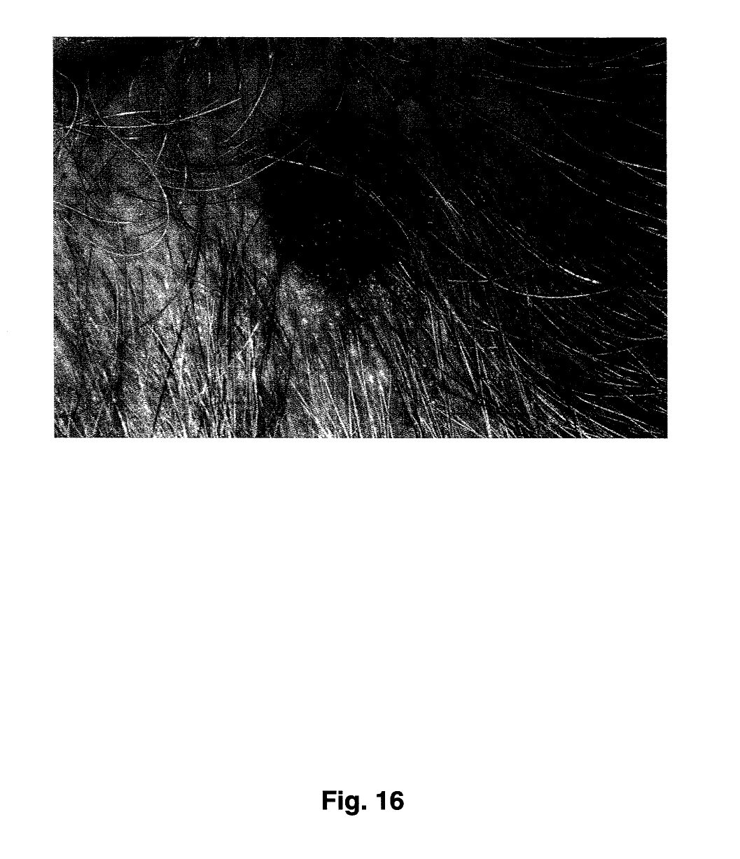

FIG. 16 shows the lesion four hours after treatment. The lesion site showed additional thrombosis and necrosis (shown as darkening), whereas the surrounding normal tissue had begun to improve in appearance.

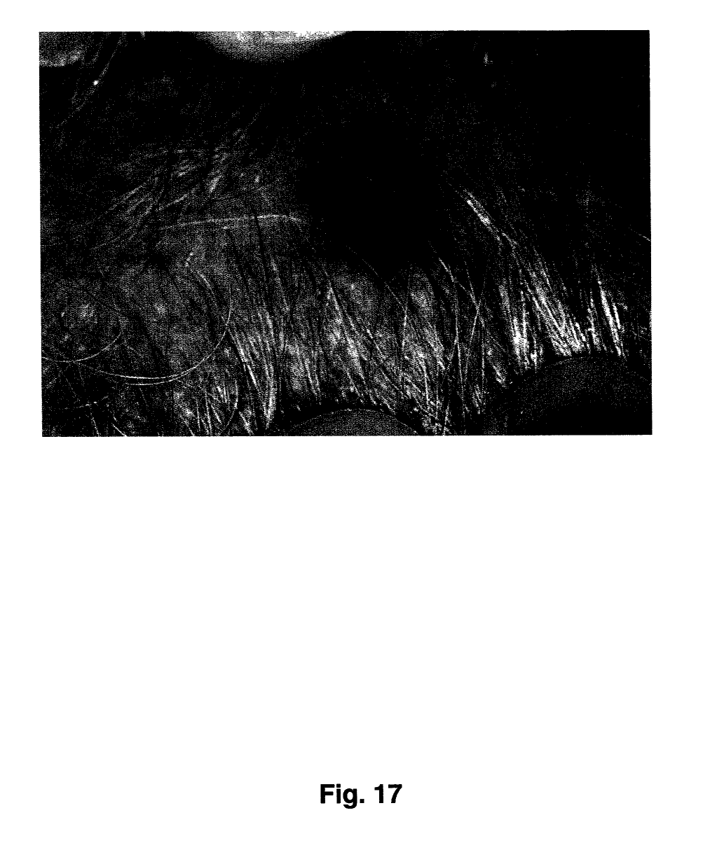

FIG. 17 shows the lesion twenty-four hours after treatment. Scar formation had begun.

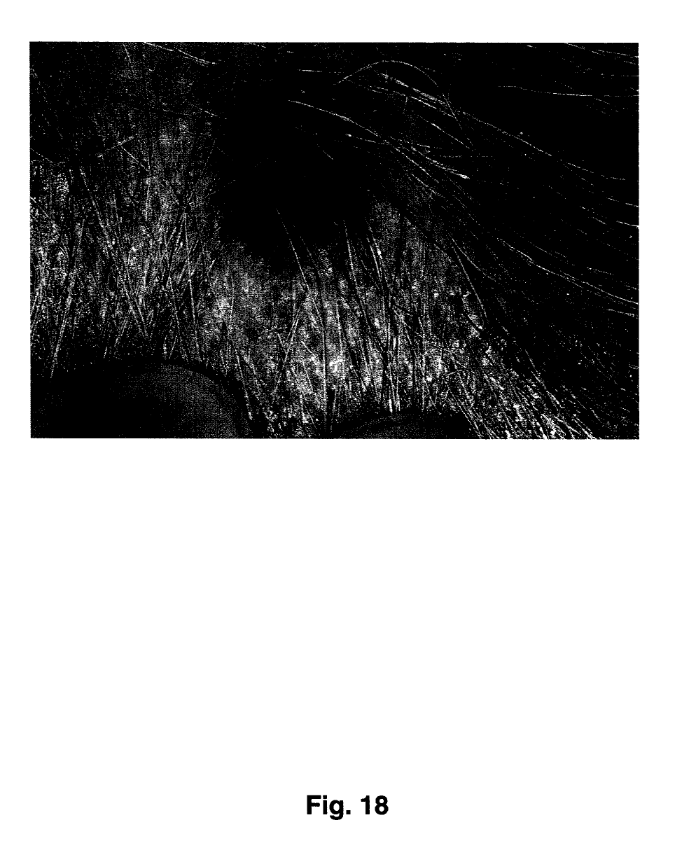

FIG. 18 shows the lesion forty-eight hours after treatment. The lesion had become completely necrotic and thrombosis was extensive. The lesion sloughed off approximately five days later.

### Example 2

### Decontamination

This example illustrates how a biological contamination, such as anthrax, is decontaminated using the present invention.

A backpack apparatus, such as that illustrated in FIG. 1A, is prepared. Two gallons of a 1 molar solution of hydrogen peroxide in water is prepared and placed in one compartment of the canister. Two gallons of 1 molar sodium hypochlorite is prepared and placed in the second compartment of the backpack canister. Both solutions are made 0.1 molar with respect to sodium dodecyl sulfate, a surfactant. The lids are screwed in place, and the compartments pressurized.

Decontamination personnel are appropriately suited, for dealing with both anthrax and the potent oxidizing agent of singlet oxygen, and the backpack is put on. A target site for decontamination, which has been tested positive for anthrax exposure, or is believed to likely be contaminated with anthrax, is sprayed with the mixture from a distance of at least 10 feet. The reactants combine to produce singlet oxygen at the target site, oxidizing any pathogen present in the area. The surfactant helps lyse any pathogenic cell and improve pathogen destruction. The mixture is left for approximately 5 minutes to decontaminate the target area.

After the reaction is complete, which is essentially immediately after application, any residue may be removed using water.

### Example 3

### Routine Disinfection

This example demonstrates how the invention is applied in a routine manner for disinfection.

The spray bottle, shown generally in FIG. 2A, is prepared. In one compartment is placed a solution of 1 molar hydrogen peroxide, and in the other compartment is placed a solution of 1 molar sodium hypochlorite. Other components in the composition may include detergents, scents, coloring agents, alcohols, etc.

The spray trigger is actuated, resulting in a sequential spray of the hydrogen peroxide solution followed by the hypochlorite solution. Upon mixing of the two solutions at the target site, singlet oxygen is produced, and a powerful oxidization effect resulting. The other components of the solution enhance the cleansing properties. The residue is then wiped up with water.

### Example 4

### Topical Antiseptic

This example demonstrates how the invention is used for topical cleansing of human skin prior to a medical treatment.

A disposable topical application bottle is prepared as shown in FIG. 3. The bottle is sized to be used in one hand and constructed from flexible plastic material. In one compartment, a 0.3 molar solution of hydrogen peroxide in reverse-osmosis water is prepared and in the other compartment, a 0.3 molar solution of sodium hypochlorite in reverse-osmosis water is prepared. The bottle is assembled with a track and a sealing tape to be removed prior to use.

When the bottle is to be used, the sealing tape is removed and the absorbent pad slid into the track. The absorbent pad is rubbed on the inner arm, where blood is to be drawn, while squeezing the bottle. The reactants simultaneously enter the pad, reacting to form singlet oxygen, which then cleanses the skin of unwanted pathogens. Several strokes are sufficient to render the skin safe for immediate injection.

### Example 5

### Wart Treatment

This example demonstrates how the invention may be used to treat a topical lesion, such as a wart.

Two solutions are prepared: 1) 0.5 M hydrogen peroxide in reverse-osmosis water, and 2) 0.5 M sodium hypochlorite in reverse-osmosis water. A topical anesthetic is applied to the wart to be treated. A volume of 0.05 ml of solution 2 is drawn into a syringe equipped with a fine gauge beveled tip hypodermic needle, and injected into the center of the 3-mm dermal wart until dermal blanching occurs. The syringe and needle are flushed with reverse-osmosis water, and the process is repeated with solution 1, taking care to inject the second solution into precisely the same area as solution 1. It is advantageous to inject or apply the hypochlorite solution first, because peroxide is immediately broken down by catalase in the body as soon as treatment begins.

Progress of the treatment is monitored by observing changes in color to the wart. Necrosis of the wart tissue is shown by changes in color to dark brown followed by black. After the color change the wart tissue sloughs off within a matter of days, and with minimal scarring.

### Example 6

### Tumor Treatment

Two solutions are prepared: 1) 0.5 M hydrogen peroxide in reverse-osmosis water, and 2) 0.5 M sodium hypochlorite in reverse-osmosis water. One milliliter of each of the solutions is poured into two separate syringes, as illustrated in FIG. 7. The conduits and catheter are attached, as illustrated in FIG. 7. Air is purged from the system.

A needle housing for the catheter is used to guide the catheter to its target site, and the needle housing is withdrawn, leaving the catheter in place. A total volume of 1 ml (0.5 ml of each) is injected into a 1-cm diameter tumor. The catheter is withdrawn after delivery of the reactants, and the site is bandaged. In an alternative, an injecting catheter may be used to both inject and deliver the components.

Progress is monitored by X-ray of the tumor over the following weeks, with progress shown by reduction in tumor size. If necessary, treatment is repeated.

The entire contents of all documents cited in this specification is a part of the present disclosure, and all documents cited herein are hereby incorporated by reference.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with a true scope and spirit of the invention being indicated by the following claims.

What is claimed is:

1. A method of treating a target site in or on a mammal, comprising: administering at least one source of peroxide and at least one source of hypochlorite anion to the target site to be treated and allowing the peroxide and hypochlorite to react to produce singlet oxygen.

2. The method according to claim 1, wherein the source of peroxide comprises at least one of hydrogen peroxide, alkyl hydroperoxides, or metal peroxides.

3. The method according to claim 1, wherein the source of hypochlorite anion comprises at least one of metal hypochlorites or hypochlorous acid.

4. The method according to claim 3, wherein the metal hypochlorites comprise at least one hypochlorite chosen from calcium hypochlorite, sodium hypochlorite, lithium hypochlorite, and potassium hypochlorite.

5. The method according to claim 1, wherein the source of hypochlorite anion source comprises chlorine dioxide.

6. The method according to claim 1, wherein the source of peroxide and source of hypochlorite anion are administered sequentially.

7. The method according to claim 6, wherein the source of peroxide and source of hypochlorite anion are administered through at least one conventional syringe and needle.

8. The method according to claim 1, wherein the source of peroxide and source of hypochlorite anion are administered simultaneously.

9. The method according to claim 8, wherein the source of peroxide and source of hypochlorite are delivered through at least one dual lumen catheter.

10. The method according to claim 1, wherein the target site is a tumor.

11. The method according to claim 1, wherein the target site is an atherosclerotic plaque.

12. The method according to claim 1, wherein the administering of at least one of the source of peroxide and the source of hypochlorite anion is performed upstream of blood flow to the target site and the blood flow carries at least one of the source of peroxide and the source of hypochlorite anion to the target site.

13. Singlet oxygen produced by a process comprising:
a) introducing into a mammal at least one composition comprising at least one source of peroxide; and
b) introducing into a mammal at least one composition comprising at least one source of hypochlorite anion.

14. A system for treating a target site in a mammal, comprising:
a) at least one source of peroxide;
b) at least one source of hypochlorite anion; and
c) at least one catheter having at least one lumen.

15. The system according to claim 14, further comprising at least one syringe and at least one conduit.

16. The system according to claim 14, wherein the target site is a tumor.

17. The system according to claim 14, wherein the target site is an atherosclerotic plaque.

18. The system according to claim 14, wherein the target site is a site of pathogenic infestation.

19. An apparatus for singlet oxygen delivery comprising:
a) a first reservoir for containing at least one peroxide source;
b) a second reservoir for containing at least one hypochlorite anion source;
c) a first conduit connecting the first reservoir to a delivery port; and
d) a second conduit connecting the second reservoir to the delivery port.

20. The apparatus according to claim 19, further comprising a mechanism to simultaneously deliver the peroxide source and the hypochlorite anion source.

21. The apparatus according to claim 19, further comprising a mechanism to control the flow of the peroxide source and the hypochlorite anion source from the first and second reservoirs through the first and second conduits to the delivery point.

22. The apparatus according to claim 19, wherein the delivery port is a catheter.

23. The apparatus according to claim 19, wherein the delivery port is a spray nozzle.

24. An apparatus for singlet oxygen delivery comprising:
a) a first reservoir for containing a composition comprising at least one peroxide source;
b) a second reservoir for containing a composition comprising at least one hypochlorite anion source;
c) a first conduit connecting the first reservoir to a first delivery port; and
d) a second conduit connecting the second reservoir to a second delivery port; wherein the first and second delivery ports are oriented to direct output to a target point.

25. The apparatus according to claim 24, wherein the at least one peroxide source and the at least one hypochlorite anion source are solutions.

26. The apparatus according to claim 25, wherein the output is a stream.

27. The apparatus according to claim 25, wherein the output is a mist.

28. The apparatus according to claim 25, wherein at least one of the compositions comprising at least one peroxide source or at least one hypochlorite anion source further comprises at least one surfactant.

29. A method for treating tumor cells or cancer cells as a result of seeding an operative site comprising:
administering as an irrigation or irrigating solution at least one source of peroxide and at least one source of hypochlorite anion.

30. A method for killing pathogens in or on a mammal comprising:
administering an aqueous solution comprising at least one source of peroxide and an aqueous solution comprising at least one source of hypochlorite anion.

31. The method according to claim 30, wherein at least one of the aqueous solutions comprising at least one peroxide source and at least one source of hypochlorite anion further comprises at least one pharmaceutically acceptable excipient.

32. A singlet oxygen producing composition comprising:
a) at least one source of peroxide;
b) at least one source of hypochlorite anion; and
c) at least one of a surfactant, detergent, scent, colorant, viscosity-modifying agent, solvent, chelator, and pH-modifying agent.

33. A method of disinfecting or decontaminating an inert area, comprising:
a) delivering at least one source of peroxide;
b) delivering at least one source of hypochlorite anion; and
c) delivering at least one a surfactant, detergent, scent, colorant, viscosity-modifying agent, solvent, chelator, and pH-modifying agent.

34. The method according to claim 33, wherein any of a), b), or c) are performed separately.

35. The method according to claim 33, wherein all of a), b), and c) are performed simultaneously.

36. A method of treating a target site in or on a mammal, comprising:
administering at least one source singlet oxygen, wherein the at least one source of singlet oxygen comprises superoxide.

37. A device for combining at least two fluid reactants, comprising:
at least a first and a second conduit for delivering separate fluid reactants;
a reaction chamber in fluid communication with said first and second conduits, wherein the reaction chamber allows for the mixing of the at least two fluid reactants; and
a reaction chamber port allowing for the passage of the at least two fluid reactants to the exterior of the device.

38. The device according to claim 37, wherein the device is a catheter.

39. The device according to claim 37, wherein the device is a hypodermic needle.

40. The device according to claim 37, wherein the device is a injecting-type or infiltrating catheter.

41. The device according to claim 37, wherein the device is a spray bottle.

42. The device according to claim 37, wherein the device is a spray canister.

43. The device according to claim 37, wherein the device is an irrigation bottle or bag.

44. The device according to claim 37, wherein the device is gravity-driven.

45. The device according to claim 37, wherein the device is pressurized.

46. The device according to claim 37, wherein the device is mechanically driven.
